SEQUENCE LISTING (162 sequences)

SEQ ID NO: 1 - DNA, 403 bp, Cannabis sativa
  misc_feature (202)..(202): n is t or a
gacacccctgatcatatctttcattcaccctattttttagttgaaacaacataaattttaaatgtggtgtgttcccaagcagtaaatgtctaaaattacagggaaacccaaaaccaattctagacgtaaatctggtgttttgggttgaagtactcaactttcgaaagggtcgactctgattgagttgactggatacacagcngcagcgattctctccctgaggttaaatttcttcgcaatcagcggtcgcacatcatcaactccaactaagctttccaaaaatggaagcaatgaaggcaactctttatccaaaggatcatcgaaccagctttcaattggtaccccattgtccacttgaaatccaaatgcctgaaagagacatagtaatcaaattttctcccaa

SEQ ID NO: 2 - DNA, 403 bp, Cannabis sativa
  misc_feature (202)..(202): n is t or c
aagattctgaatgtacaatgagagattaaaatcttgagatcatcttcgcctccaccagtctggaatttttagcctttggtttctatgtacagcaagtttaaccccaggaagcttatgtttcacctgtctccataacatatcaactgcatcttcctttttcggtgggtactggaattcaaaatgatgcgaaatgttcttaagntgtctccacatttcaacccatttttccttgggaaattcacggagcttagtaaccatgtaaccaggttccagagcctctttaaatgagaagaataatgagaactggctgtagtcaatttcatcctcgaatgggagctcaatttgatcactcactatgacaggaatacaatgactcacaatagcatcaaataaacgacatgat

SEQ ID NO: 3 - DNA, 403 bp, Cannabis sativa
  misc_feature (202)..(202): n is t or c
aacatgctaattctgatttaaagctgcatatttggtttagagatatattacctgggcaagtttcagatttgtacatttaaagcaaatctggtggataatgtaaaaggaaatttgaaatacattgtaactccagtctgtgaaactcacattttacattgacatgtaatgctctttgttttcatacttgaaggaacaagaaggngcgactttgcttgtatctgcaaaatttgatgtagtttctgtccgtaatatctaccttcagtttgaagaggtatgtttttcttcttttgtgaagcatgactttcaaaaatttcaagtttaaatgtgtgtggttgttccctctacatggttctctgtttgatgtaccattacttaatctggcctgctctattgagtcatattt

SEQ ID NO: 4 - DNA, 403 bp, Cannabis sativa
  misc_feature (202)..(202): n is to ro g
ttcggagttggaccaagatgaatgtagtgtttatttgcagcagagtagccctcctattaactcaatcactggcttttctggtatggtttttctatcactaggaatcatgcttgacccaatttatgggcagacaagttatatttatcttgtctaacataaaatattctaactggctcagtttcactcggagcaattacatctnctgccgtagataatgggagtactatagctgctcagagtgcaacacaaaatccatccctggaagctgcatttcatcacgggatatcttctagtgttcctaacagcttatcctctctagttagaattgaatctctaggcaatcatgctggcctttcagaatccaatcattcatcggggccactaaagtttgacatccatggaa

SEQ ID NO: 5 - DNA, 403 bp, Cannabis sativa
  misc_feature (202)..(202): n is t or a
ctgaccttttcttttttccttctttccggtgtatgaaggtggaaaccattatcaagaaaatggaagttactggaggctaaaaccatcttggttttatggcactgatcggtatgtttttctttttctaagactcttaatctccatcaatactgatgaatttatacagcttttatttatttattatgctactgtgtttgttttnataggactttggcaaatgtgtggagggcgagacgctactgttatcttacaaattagtttaaataaaagaacaagaatcagtttttctaagagcagaacctctggtatggaatttgaatcagagcggaggaaacacaagaaatcgttggcatttaccactgaagttattcgttctgcaacattttttatggcttggtcttgt

SEQ ID NO: 6 - DNA, 403 bp, Cannabis sativa
  misc_feature (202)..(202): n is t or c
ttcagattaaagccggtgccaagctttttctttacgactttgatgtgaagcttctttatggtgtctacgaggccacttcagttggtgctctcaacttggaacccactgcctttcatggaaaattccctgcccaggtacctcccctcttcttcgtcttcttctttaagggtcggttttgttttgattccttgttcgtttgttnaggtcaagttcaagattttcaaggaatgtttacctcttcccgagagggttttcaaagctgcaattattgacaattaccagggttcaaggtttaaacaactacttagtagtgcacaggtaaagctactacttgatttgtaatctcgtttatcattattattaattgagtttatttctttctcaattcaattcaatgaaggtg

SEQ ID NO: 7 - DNA, 403 bp, Cannabis sativa
  misc_feature (202)..(202): n is a or g
gccattaagcccaatgcctccatggactgccattttccgagcaccaatgcagcatgctgcatgagccccacggtggggcgggacaatggatcccacatcaagcagcttccatgacaatgttataccgagagtctcgtggcatgataattgtccaatccatgtgtcattcatacggttcccatgatcatcaattcctccaaanacaactagaagttcaccaattacaacacatgaatgtccaaatctcccacttggaatgcctgaattaagcttctgccacttcaactttctttgacaatcattaccaatatatgccacccatgtgtcatcaagatggcgtcctaaacagtaaaagagaaaagagaaacaaaatcaatgcataacaaaaagaaaattaagagca

SEQ ID NO: 8 - DNA, 403 bp, Cannabis sativa
  misc_feature (202)..(202): n is a or g
tcttcaatcttcaatcatagtagagaataatagatacaacataaatattatggcttgttttgtgtttggtgtatgatgataataatgatgtttgacgattaataaaacacaagcctaaaatggagttgagagagtgatcatgatggaaataatattaatcaatcaaacatctctgtcacgtttgcactcaggtgacatgttngggtatctgacacgatcagtgcaatagttgtaaatggtgtacttttgtctgacccacctgagatacctccactggctctggtcaaggtcctggaactccttaccatcccaccacctctttccttgggtggcacagtacttggcttgaaccgatgactcgcacccatcgatgtggaaccctctgtaggccgctatgaatggg

SEQ ID NO: 9 - DNA, 403 bp, Cannabis sativa
  misc_feature (202)..(202): n is c or t
tattttgatttgggaatttttgattatttgttctgacaaatttgaaatcttcgttatggagcaggaatcaagcaaagtgttaagcatgtctagagttcgctgcattctccgtggtttggatgtgaaaactcttgtctttctctttgcccttatcccaacttgcatctttttcatctatgttcacggacagaagatctcatanttcttgcggccactgtgggaatcaccacctaaaccttttcatgatatgccgcactattatcatgagaatgtgtcaatggaacatctttgtaaacttcatggttggggagtgagggagtatcctaggcgtgtttatgatgctgtgttgtttagtaatgagctagacatcttgaccattcgctggaaagagttgtatccctac

SEQ ID NO: 10 - DNA, 403 bp, Cannabis sativa
  misc_feature (202)..(202): n is c or t
acttctaaaaatggcggcatcttcaagactagtgctgcatctacatgccacaaccaccgcagctatagtggtgcctacacccaagtacaaccttagattatccaccgccacagctgctaatcgccgctttcgaaaacccatattcaaatgtaaggctacctctaacactactcctacttctactcctgttttccaaggaatntacggtccttggtccgtcgattccaccgacgttagagaggtcatatcctaccgttctgggctggtcacagctgcagcctcttttgttggggcagcctccacagctttcttgcctgaagaaaatcaggtcggggaattcatacaccacaatcttgacctgttttacattgtgggtggtgctggacttgggttgtctttggct

SEQ ID NO: 11 - DNA, 403 bp, Cannabis sativa
  misc_feature (202)..(202): n is g or t
tcatggttgtgtgattaaattttaataattaaataaatactatatttgatgtgattactaaattggatcaacatatcacctacatatagtttgtatgtttaaaaaattaatactagagaaattagataggagagatataattttaatgtaaatgtgtacctgatagcttccaataacatggatgacgacaaacatattagcngaagcaataagccaagcaggcttttcaagggtgatgagaatattgtcatctaccgagttaccaaaaacataataacctatcaaagcaactggaaaataacaaagagctactactatgtaagccacaactactcctctccacattggtttcttagatggtttttctggtgtggatgggattgtggcttgaatctcaagcacc

SEQ ID NO: 12 - DNA, 403 bp, Cannabis sativa
  misc_feature (202)..(202): n is a or t
ctcaaaactccactttctgctgctcgacatgttatcattgaaacccactactacacctctcaacaacaaccccaacatctccggcgaccggagtttcagctcgatctccaccgccaccgccgggaaagatggagaccttagaagaaagacccgcgtggcggtttctgggtcgaaactcaggcgacgtgggtcggttcgggcngcgatcagtagtggggacaacaaaacagagactgtgagtagtaatagctctgttccggctcatcagagtgaagataattccaacggttctctgaagaagaagaagccatctaaaggaattgaagttagagcagtgatgactatcaggaagaagatgaaggagaagctcgctgaaaaaatggaggatcaatgggagtttttc

SEQ ID NO: 13 - DNA, 403 bp, Cannabis sativa
  misc_feature (202)..(202): n is c or t
cacaatattacaacatgtacagtttgtgctataagtttctatcttttttcttcttcttctttttcctttatttttaggccaaaactaaacatggtagatcatccccacctcgagagtggaagctcggggcacgtttagatcataagtggctcctctgctgtttcttctcaagaactcgtatccataatcgattgcaatcctnttcgttatacctgatccgggatttgctctcatgtaagtgttccctaatatgtaagctattccggtttccttagcttccatcaactctgcgagttctgccctcatactcgtgtcaatcttgggactctccggcaaaacaaacctcaccttcttttttcttggaatagctggtggtgatggtgatgttatctctctatgattt

SEQ ID NO: 14 - DNA, 403 bp, Cannabis sativa
  misc_feature (202)..(202): n is a or g
gaaaatatgtggtttttttgtgtataactttgtttagtatctccaagatcctatgttggtctttaacaaagaaacatatagtaactcattccaagtatctggaacaccaggcaaacaccaatggctgcaatcttgagaatgtacagcagcaatttgctcctctactgatttatattccattcgataaatcgaagggtgaccntcttttctgtaatctgtaagcctgcttatgtttagatatgtcactggagttttcatatcttgaatcacatactctaaagccctcatctttttattgtacttagctaaataagtgttgttgaaaatcggctcggtttctttgtggcattgtcctcctgagttccatggccctcctctgccaacaagattaaagttcaacatt

SEQ ID NO: 15 - DNA, 403 bp, Cannabis sativa
  misc_feature (202)..(202): n is c or t
aattctgataattacttagcacatagagaataataagaattgccagaaatgttgcccatgtttgatccaaacgacaatgaagctggtatgaagcttttggaggacctaaccacaaatgcacaccattttcaacaacaggcactgaaggagatactatcaaacaatgctgccactgaatatctaagcagctttctcaatggtnactctgatatgaagcttttcaaggaaagagttcccattgtgaagtatgaagatatcaagccttttatcaaccgaattgccaatggggaatcctccaacatcatttcagctcaaccaataacagagcttcttacgaggtataacatcacatatatatatatatatatatatatgatatatgacatgacatgttatgacacat

SEQ ID NO: 16 - DNA, 403 bp, Cannabis sativa
  misc_feature (202)..(202): n is g or a
tagatatacttgaataatataccgtgctccatgtatagctagctctttcattctggctataaactaattgaggggataatacatatattatatatacatattttgtatacttatcttctttcattgttaacgaaaatgaggattagggatgtgttattgggtgcattggtgagctatctgatcatacaaaacgtttgtgtancaaatgcatcgagggttttacaaaagtcacagttttttgccaactttttgcaatctaataataatgggattagtaataatgggaccaaatgggcagttcttgttgctggctccaatggctggggtaactacaggcatcaggtatttaatgggacaccataaacacgggagggagtatatataaatatacatatatatatat

SEQ ID NO: 17 - DNA, 403 bp, Cannabis sativa
  misc_feature (202)..(202): n is a or c
tatttcacaacaaaaagacacgagaaaaattgtctaatcaaatcaaattccagtatcatgctagtattaagtgaatcaaaaaatcaagaggcatataatatataatagagaactaagagaagcacaataaaagaaatatcagcacaatgatagttagagtagctaaatttgagaacatatcagatgtcaatgaaaatagganattggcgcaccttctccacatagctgttcaaatcgagcaactatgtgttttccatacgtatatttcctcagagcagcaagatgggctcttgtgcgattcagcaatattgctcgctgtctgtcattgcttttctcaaacatcttttgcaccacataattagcaaattggtccttcatcattgtctagatataggaagcaaac

SEQ ID NO: 18 - DNA, 403 bp, Cannabis sativa
  misc_feature (202)..(202): n is g or t
aaaggcaaggattgaaatgagcagaacaggaccagtgaacaacaagaagaagcttcctgcaaattatagcaccaagtataaagaacgcaaaagttccatcaaataaaaaaaaatagtgtgaaaagggacagaaaatttggaaatcaaacctgtaaacccaaaaacagttccaccagttgcattgaacaatacagtgatagtntgagttgaaaactttcctggcaacaaaaatgcaataccaacccataaaataaaaacgacccacattagaactttgagtatgaatttggccaaagaggtaaaaagagatgttttcctcttgacataactaggctccacagtcggaggcaacagaagaggcttctcaacagagtttccgtccatggcgaaaaccttacacaat

SEQ ID NO: 19 - DNA, 403 bp, Cannabis sativa
  misc_feature (202)..(202): n is g or a
ccatccgacactcctcaacgttctgaggaatggtttgcccttcgtaaggacaagctaaccacaagcactttcagcactgcattgggtttttggaaaggacagcgtcgaatggagctttggcgcgagaaggtgtttgcatcagaggtcaaaatcatacaaggtgcacaaagatttgctatggattggggtgttctcaatgaancagaagctatagaaaggtacaaaagcattacaggccgggaagttgattcgctaggatttgctgttcatgctgaggagcgatacaattgggttggcgcctctcctgatggtgttattggatgcttcccggagggtggaattctggaagtgaagtgtccttataacaagggtaagcctgagttgggattgccttggtctaaaa

SEQ ID NO: 20 - DNA, 403 bp, Cannabis sativa
  misc_feature (202)..(202): n is t or c
gtcaatatttttaatttttatgtatacataatattagatttagtatgcaaattcatgtaagttattattaatttagacaattatatatatttatatatatatatatatgatatgttcttacactaattgaagagccatggtgggatcttggcgtgagtagtgattgttatttggttgtaaagcattgacttggaaataattncctggttgctggtgtaccacaactgatgctgcaccctcatcatcattattattattattattattattatgatcattgttattattatgattttgatgaattagttcatgatgatgaccatagctgcttgtaccaacattcatgttcatatttatgttcatgttcatattcatactcatgctttggttcctctcattctca

SEQ ID NO: 21 - DNA, 403 bp, Cannabis sativa
  misc_feature (202)..(202): n is c or t
gcttaaaaatctatatattgaaaaaaaaaaaaacatgaattaaaatttataattacagggatgtaacattttcttattgataaatcccaaagtttcaatttttttttttaattttaaattttctcattctaataataaaaaataaaaaataaaaacttttaattatgcttccaagaaaccagtcattctaagaatagaattnctaacatgactcaaatctctccccttaagagttcttccattgcccgaacaaaccgaaaaagccatctttccaccggttaaccgcctcctgacgatgacatgaggcggtaccagttcttcattttcatcataatcataatcataatcataattatcatcatcgaaattattccaaatttcgtgatttaaatgatcattaata

SEQ ID NO: 22 - DNA, 403 bp, Cannabis sativa
  misc_feature (202)..(202): n is c or t
ttgatacagaagtttgtcaaagtagtttacatacatacatacatacattgataaaggaaaagctatagtagtagagccatgaaaactggtagtaacactggggatttaccgggttatatcaggcgtttcaaacccgtgatagaagcattcggtttttcctcgaaaagttcaagttcaagtcatcctgcttaaatctactctngttcctcctccccattgtcgttctttcctcgttttgaattctacgtcgataaggaagtaacatttgtcattctctctaatgtacttgatctgtccattcattctactgagaagttttcgagaaaggtttaacccgagcccttcttgtgaagtccagtgctttccactctcaaccatgtcttggataagagcattaggaata

SEQ ID NO: 23 - DNA, 403 bp, Cannabis sativa
  misc_feature (202)..(202): n is a or g
atagaagcattacttcttgcttctgagtcgagaatcgaaaagtccagcaaagaaattgatctcagtgccaacttggtcaccaatgatctggactccacggctgaagctaatcttgcatttagaagattcaagcaatttggtcgtggcaattctcaactcagcaatgcttctgctcaatttaacaggattcctaatcctaatntcaggctcaacaatttttctcccaatcccaatcagagtagcatgagcagaggtaatttcaattttaatcctctcaaaaataacaggtttggatttacctttcctaaccggcctcagtgccaactctgcttgcgatttggtcatgttgtgcaagattgtccctttcgttttgacaaatctttctcaggaccacctttagcta

SEQ ID NO: 24 - DNA, 403 bp, Cannabis sativa
  misc_feature (202)..(202): n is a or g
tttttgtcgttgaaaattctccctgaactataattaagtgattagcattgcattaggcatcaggaaggtgatcatctctctccgtaccttttatttctctattatgagggtcttactagtgcccttaagattcatgaaaggcttggtacgtagtctcatgggtatctttgttgctcgcactgcctctgccatttctcacttnctttttgatgatgacaatctcctattcaccactgctacccatacttctttcaatgctttggagaatgcccttattctttataacctagcctctggtcaaaaggtttattatgggaagtcttccattttgttctcccccaacactcatccatccatcttgagctacttttatgaaaccttggggttgaattctaagctcttt

SEQ ID NO: 25 - DNA, 403 bp, Cannabis sativa
  misc_feature (202)..(202): n is c or t
ctgtcccatagcttactactccagatcagaaaaaccaggtgtgagctacaaatactacccaactgtgaaagagttggctgccaactccgacattttggtggttgcttgtgcactcactgaggaaacccgccacattgtcaaccgtgaagtcatcgacgcattgggcacaaagggtgttctcatcaacatcgggaggggtccncatgtcgacgaacctgagctagtatcagccctggttgaaggccgattagggggcgctggccttgatgtctaccaaaatgagcctgaggttcctgaggagctatttggtcttgaaaacgttgtccttttgcctcatgttggaagtggcactatcgaaacacgccaggccatggctgatctggtggttggtaaccttgaagct

SEQ ID NO: 26 - DNA, 403 bp, Cannabis sativa
  misc_feature (202)..(202): n is t or c
tgtgatttgaaaaccagaagtgttgttggatctcaacacagaaatgatttgtgggtctgtgtgttcaccaaacccaatcaaattcctaccactcaaagctccaagctctggacatggtggataatgattgagtctgaaacacgagtcacttttctcatcactcaacattttactcagtacatttctcggttcaattcttaanccatcagccattaattcaagtatctcaaacgacatcgttttaacagccgttatatatttctccaccgccgaactattcaaaaacgacaaccaattagtcaaaacgacaaccaatatcaaaaacagagtaaaaaaaaaaaatgaaaaaacagagtttttattttaccggaaaatttcaggattttctcggaaagtgaagagg

SEQ ID NO: 27 - DNA, 25 bp, Artificial sequence
  Primer
gcggagtttggatttgaaggtggaa

SEQ ID NO: 28 - DNA, 25 bp, Artificial sequence
  Primer
taactcaaccctacgattcgccaaa

SEQ ID NO: 29 - DNA, 22 bp, Artificial sequence
  Primer
ctcaaccctacgattcgccaac

SEQ ID NO: 30 - DNA, 103 bp, Cannabis sativa
  misc_feature (52)..(52): n is g or t
ggcggatgtgggcggagtttggatttgaaggtggaaaaagttggagaggganttggcgaatcgtagggttgagttattattggtggagaatggacggtggaga

SEQ ID NO: 31 - DNA, 24 bp, Artificial sequence
  Primer
gcagctcattgagatgacacccaa

SEQ ID NO: 32 - DNA, 24 bp, Artificial sequence
  Primer
tgtggatgcttccatggtcgtaca

SEQ ID NO: 33 - DNA, 22 bp, Artificial sequence
  Primer
tggatgcttccatggtcgtacg

SEQ ID NO: 34 - DNA, 103 bp, Cannabis sativa
  misc_feature (52)..(52): n is g or a
atatgtgtaggctatcattgtgtcatgctgtggatgcttccatggtcgtacnttgggtgtcatctcaatgagctgcgacaatgaggcaacccgaggttttgga

SEQ ID NO: 35 - DNA, 29 bp, Artificial sequence
  Primer
agtgacattggattgatcattctgcgaat

SEQ ID NO: 36 - DNA, 23 bp, Artificial sequence
  Primer
tagaaagagggtaccactgccat

SEQ ID NO: 37 - DNA, 23 bp, Artificial sequence
  Primer
tagaaagagggtaccactgccac

SEQ ID NO: 38 - DNA, 103 bp, Cannabis sativa
  misc_feature (52)..(52): n is g or a
tggtggttccattattagtgacattggattgatcattctgcgaatcagtggntggcagtggtaccctctttctaccaaacttggcatcataaaacattttaaa

SEQ ID NO: 39 - DNA, 26 bp, Artificial sequence
  Primer
ccttcggaatcaaggagaaggatgtt

SEQ ID NO: 40 - DNA, 22 bp, Artificial sequence
  Primer
ctgtttcttttggcaggaggcg

SEQ ID NO: 41 - DNA, 23 bp, Artificial sequence
  Primer
cctgtttcttttggcaggaggca

SEQ ID NO: 42 - DNA, 103 bp, Cannabis sativa
  misc_feature (52)..(52): n is a or g
ttgttttgagatttttaatttttcgtttgcctgtttcttttggcaggaggcntgcccgtctgtgaaaaacatccttctccttgattccgaaggaaagcgtgtt

SEQ ID NO: 43 - DNA, 27 bp, Artificial sequence
  Primer
gacatatgggatgtggatgtttgggaa

SEQ ID NO: 44 - DNA, 32 bp, Artificial sequence
  Primer
tcatttttgttgtttcgaaatgaaactttcag

SEQ ID NO: 45 - DNA, 36 bp, Artificial sequence
  Primer
gctatcatttttgttgtttcgaaatgaaactttcaa

SEQ ID NO: 46 - DNA, 103 bp, Cannabis sativa
  misc_feature (52)..(52): n is t or c
agatatttggttgtgttggatgacatatgggatgtggatgtttgggaagcantgaaagtttcatttcgaaacaacaaaaatgatagccgagtaatgatcacaa

SEQ ID NO: 47 - DNA, 27 bp, Artificial sequence
  Primer
cacaagaggtacaacaaccacaaccat

SEQ ID NO: 48 - DNA, 31 bp, Artificial sequence
  Primer
ttcttaaactgtttagtgatcaattgatgga

SEQ ID NO: 49 - DNA, 29 bp, Artificial sequence
  Primer
cttaaactgtttagtgatcaattgatggg

SEQ ID NO: 50 - DNA, 103 bp, Cannabis sativa
  misc_feature (52)..(52): n is c or t
aacggtaggagaaaacccggaaccacaagaggtacaacaaccacaaccatcnccatcaattgatcactaaacagtttaagaactaatgagatatctgatgatg

SEQ ID NO: 51 - DNA, 29 bp, Artificial sequence
  Primer
ggagtactcttatcttttggatcaagcat

SEQ ID NO: 52 - DNA, 33 bp, Artificial sequence
  Primer
gttttcatagttttaataacacttacatctttt

SEQ ID NO: 53 - DNA, 33 bp, Artificial sequence
  Primer
gttttcatagttttaataacacttacatctttc

SEQ ID NO: 54 - DNA, 103 bp, Cannabis sativa
  misc_feature (52)..(52): n is c or t
aaaaaaaagctatcataatgttttcatagttttaataacacttacatctttnccatgttggtatgcttgatccaaaagataagagtactccaattcttcacaa

SEQ ID NO: 55 - DNA, 25 bp, Artificial sequence
  Primer
tcacctgagggatttccgcaacata

SEQ ID NO: 56 - DNA, 26 bp, Artificial sequence
  Primer
cagtgaagcaaactaatcctcgtcaa

SEQ ID NO: 57 - DNA, 25 bp, Artificial sequence
  Primer
agtgaagcaaactaatcctcgtcag

SEQ ID NO: 58 - DNA, 103 bp, Cannabis sativa
  misc_feature (52)..(52): n is c or t
aatgacgcgaattgaggtttccatactcacctgagggatttccgcaacatantgacgaggattagtttgcttcactgacaattgacaatcctaattcaacaca

SEQ ID NO: 59 - DNA, 20 bp, Artificial sequence
  Primer
atgtggtgtgttcccaagca

SEQ ID NO: 60 - DNA, 20 bp, Artificial sequence
  Primer
tttcaagtggacaatggggt

SEQ ID NO: 61 - DNA, 20 bp, Artificial sequence
  Primer
atgtggtgtgttcccaagca

SEQ ID NO: 62 - DNA, 20 bp, Artificial sequence
  Primer
tcaagtggacaatggggtac

SEQ ID NO: 63 - DNA, 20 bp, Artificial sequence
  Primer
catcttcgcctccaccagtc

SEQ ID NO: 64 - DNA, 20 bp, Artificial sequence
  Primer
tgagctcccattcgaggatg

SEQ ID NO: 65 - DNA, 20 bp, Artificial sequence
  Primer
atcttcgcctccaccagtct

SEQ ID NO: 66 - DNA, 20 bp, Artificial sequence
  Primer
tgagctcccattcgaggatg

SEQ ID NO: 67 - DNA, 22 bp, Artificial sequence
  Primer
acctgggcaagtttcagatttg

SEQ ID NO: 68 - DNA, 20 bp, Artificial sequence
  Primer
agagggaacaaccacacaca

SEQ ID NO: 69 - DNA, 22 bp, Artificial sequence
  Primer
cctgggcaagtttcagatttgt

SEQ ID NO: 70 - DNA, 20 bp, Artificial sequence
  Primer
agagggaacaaccacacaca

SEQ ID NO: 71 - DNA, 20 bp, Artificial sequence
  Primer
tttgcagcagagtagccctc

SEQ ID NO: 72 - DNA, 20 bp, Artificial sequence
  Primer
tcccgtgatgaaatgcagct

SEQ ID NO: 73 - DNA, 20 bp, Artificial sequence
  Primer
tcggagttggaccaagatga

SEQ ID NO: 74 - DNA, 20 bp, Artificial sequence
  Primer
tcccgtgatgaaatgcagct

SEQ ID NO: 75 - DNA, 20 bp, Artificial sequence
  Primer
ttccggtgtatgaaggtgga

SEQ ID NO: 76 - DNA, 21 bp, Artificial sequence
  Primer
tccataccagaggttctgctc

SEQ ID NO: 77 - DNA, 20 bp, Artificial sequence
  Primer
tccggtgtatgaaggtggaa

SEQ ID NO: 78 - DNA, 21 bp, Artificial sequence
  Primer
tccataccagaggttctgctc

SEQ ID NO: 79 - DNA, 20 bp, Artificial sequence
  Primer
tcagattaaagccggtgcca

SEQ ID NO: 80 - DNA, 21 bp, Artificial sequence
  Primer
gcagctttgaaaaccctctcg

SEQ ID NO: 81 - DNA, 20 bp, Artificial sequence
  Primer
gattaaagccggtgccaagc

SEQ ID NO: 82 - DNA, 21 bp, Artificial sequence
  Primer
gcagctttgaaaaccctctcg

SEQ ID NO: 83 - DNA, 20 bp, Artificial sequence
  Primer
taagcccaatgcctccatgg

SEQ ID NO: 84 - DNA, 20 bp, Artificial sequence
  Primer
ttcaggcattccaagtggga

SEQ ID NO: 85 - DNA, 20 bp, Artificial sequence
  Primer
gccattaagcccaatgcctc

SEQ ID NO: 86 - DNA, 20 bp, Artificial sequence
  Primer
ttcaggcattccaagtggga

SEQ ID NO: 87 - DNA, 22 bp, Artificial sequence
  Primer
tggcttgttttgtgtttggtgt

SEQ ID NO: 88 - DNA, 20 bp, Artificial sequence
  Primer
ccaagtactgtgccacccaa

SEQ ID NO: 89 - DNA, 22 bp, Artificial sequence
  Primer
tggcttgttttgtgtttggtgt

SEQ ID NO: 90 - DNA, 20 bp, Artificial sequence
  Primer
aagtactgtgccacccaagg

SEQ ID NO: 91 - DNA, 20 bp, Artificial sequence
  Primer
tgcattctccgtggtttgga

SEQ ID NO: 92 - DNA, 22 bp, Artificial sequence
  Primer
gggatacaactctttccagcga

SEQ ID NO: 93 - DNA, 20 bp, Artificial sequence
  Primer
agagttcgctgcattctccg

SEQ ID NO: 94 - DNA, 21 bp, Artificial sequence
  Primer
ccagcgaatggtcaagatgtc

SEQ ID NO: 95 - DNA, 20 bp, Artificial sequence
  Primer
agattatccaccgccacagc

SEQ ID NO: 96 - DNA, 20 bp, Artificial sequence
  Primer
ccagcaccacccacaatgta

SEQ ID NO: 97 - DNA, 20 bp, Artificial sequence
  Primer
gattatccaccgccacagct

SEQ ID NO: 98 - DNA, 20 bp, Artificial sequence
  Primer
ccagcaccacccacaatgta

SEQ ID NO: 99 - DNA, 20 bp, Artificial sequence
  Primer
ccaagcaggcttttcaaggg

SEQ ID NO: 100 - DNA, 20 bp, Artificial sequence
  Primer
ccacaatcccatccacacca

SEQ ID NO: 101 - DNA, 20 bp, Artificial sequence
  Primer
agcaggcttttcaagggtga

SEQ ID NO: 102 - DNA, 20 bp, Artificial sequence
  Primer
ccacaatcccatccacacca

SEQ ID NO: 103 - DNA, 20 bp, Artificial sequence
  Primer
gaccggagtttcagctcgat

SEQ ID NO: 104 - DNA, 20 bp, Artificial sequence
  Primer
ttcagcgagcttctccttca

SEQ ID NO: 105 - DNA, 20 bp, Artificial sequence
  Primer
accggagtttcagctcgatc

SEQ ID NO: 106 - DNA, 20 bp, Artificial sequence
  Primer
ttcagcgagcttctccttca

SEQ ID NO: 107 - DNA, 20 bp, Artificial sequence
  Primer
atcatccccacctcgagagt

SEQ ID NO: 108 - DNA, 20 bp, Artificial sequence
  Primer
catcaccatcaccaccagct

SEQ ID NO: 109 - DNA, 20 bp, Artificial sequence
  Primer
agatcatccccacctcgaga

SEQ ID NO: 110 - DNA, 20 bp, Artificial sequence
  Primer
catcaccatcaccaccagct

SEQ ID NO: 111 - DNA, 20 bp, Artificial sequence
  Primer
caccaggcaaacaccaatgg

SEQ ID NO: 112 - DNA, 20 bp, Artificial sequence
  Primer
atcttgttggcagaggaggg

SEQ ID NO: 113 - DNA, 20 bp, Artificial sequence
  Primer
ctggaacaccaggcaaacac

SEQ ID NO: 114 - DNA, 20 bp, Artificial sequence
  Primer
atcttgttggcagaggaggg

SEQ ID NO: 115 - DNA, 20 bp, Artificial sequence
  Primer
tgccagaaatgttgcccatg

SEQ ID NO: 116 - DNA, 20 bp, Artificial sequence
  Primer
ttggaggattccccattggc

SEQ ID NO: 117 - DNA, 20 bp, Artificial sequence
  Primer
gccagaaatgttgcccatgt

SEQ ID NO: 118 - DNA, 20 bp, Artificial sequence
  Primer
ttggaggattccccattggc

SEQ ID NO: 119 - DNA, 20 bp, Artificial sequence
  Primer
accgtgctccatgtatagct

SEQ ID NO: 120 - DNA, 20 bp, Artificial sequence
  Primer
ccattggagccagcaacaag

SEQ ID NO: 121 - DNA, 20 bp, Artificial sequence
  Primer
accgtgctccatgtatagct

SEQ ID NO: 122 - DNA, 20 bp, Artificial sequence
  Primer
tggagccagcaacaagaact

SEQ ID NO: 123 - DNA, 23 bp, Artificial sequence
  Primer
tttcacaacaaaaagacacgaga

SEQ ID NO: 124 - DNA, 20 bp, Artificial sequence
  Primer
tgaatcgcacaagagcccat

SEQ ID NO: 125 - DNA, 23 bp, Artificial sequence
  Primer
tcacaacaaaaagacacgagaaa

SEQ ID NO: 126 - DNA, 20 bp, Artificial sequence
  Primer
tgaatcgcacaagagcccat

SEQ ID NO: 127 - DNA, 23 bp, Artificial sequence
  Primer
gcttcctgcaaattatagcacca

SEQ ID NO: 128 - DNA, 20 bp, Artificial sequence
  Primer
tccgactgtggagcctagtt

SEQ ID NO: 129 - DNA, 22 bp, Artificial sequence
  Primer
agcaccaagtataaagaacgca

SEQ ID NO: 130 - DNA, 20 bp, Artificial sequence
  Primer
aagcctcttctgttgcctcc

SEQ ID NO: 131 - DNA, 20 bp, Artificial sequence
  Primer
catccgacactcctcaacgt

SEQ ID NO: 132 - DNA, 20 bp, Artificial sequence
  Primer
ggcgccaacccaattgtatc

SEQ ID NO: 133 - DNA, 20 bp, Artificial sequence
  Primer
ccatccgacactcctcaacg

SEQ ID NO: 134 - DNA, 20 bp, Artificial sequence
  Primer
ggcgccaacccaattgtatc

SEQ ID NO: 135 - DNA, 20 bp, Artificial sequence
  Primer
tgaagagccatggtgggatc

SEQ ID NO: 136 - DNA, 22 bp, Artificial sequence
  Primer
tgagaggaaccaaagcatgagt

SEQ ID NO: 137 - DNA, 20 bp, Artificial sequence
  Primer
gagccatggtgggatcttgg

SEQ ID NO: 138 - DNA, 22 bp, Artificial sequence
  Primer
tgagaggaaccaaagcatgagt

SEQ ID NO: 139 - DNA, 20 bp, Artificial sequence
  Primer
tgcttccaagaaaccagtca

SEQ ID NO: 140 - DNA, 20 bp, Artificial sequence
  Primer
gaactggtaccgcctcatgt

SEQ ID NO: 141 - DNA, 20 bp, Artificial sequence
  Primer
tgcttccaagaaaccagtca

SEQ ID NO: 142 - DNA, 20 bp, Artificial sequence
  Primer
aactggtaccgcctcatgtc

SEQ ID NO: 143 - DNA, 20 bp, Artificial sequence
  Primer
aacactggggatttaccggg

SEQ ID NO: 144 - DNA, 21 bp, Artificial sequence
  Primer
tgagagtggaaagcactggac

SEQ ID NO: 145 - DNA, 20 bp, Artificial sequence
  Primer
cactggggatttaccgggtt

SEQ ID NO: 146 - DNA, 21 bp, Artificial sequence
  Primer
tgagagtggaaagcactggac

SEQ ID NO: 147 - DNA, 20 bp, Artificial sequence
  Primer
tcagtgccaacttggtcacc

SEQ ID NO: 148 - DNA, 20 bp, Artificial sequence
  Primer
aaatcgcaagcagagttggc

SEQ ID NO: 149 - DNA, 20 bp, Artificial sequence
  Primer
agtgccaacttggtcaccaa

SEQ ID NO: 150 - DNA, 20 bp, Artificial sequence
  Primer
aagcagagttggcactgagg

SEQ ID NO: 151 - DNA, 20 bp, Artificial sequence
  Primer
aggcatcaggaaggtgatca

SEQ ID NO: 152 - DNA, 20 bp, Artificial sequence
  Primer
tggatgagtgttgggggaga

SEQ ID NO: 153 - DNA, 22 bp, Artificial sequence
  Primer
ggcatcaggaaggtgatcatct

SEQ ID NO: 154 - DNA, 20 bp, Artificial sequence
  Primer
ggatggatgagtgttggggg

SEQ ID NO: 155 - DNA, 20 bp, Artificial sequence
  Primer
gcttgtgcactcactgagga

SEQ ID NO: 156 - DNA, 20 bp, Artificial sequence
  Primer
ccaaccaccagatcagccat

SEQ ID NO: 157 - DNA, 20 bp, Artificial sequence
  Primer
gcttgtgcactcactgagga

SEQ ID NO: 158 - DNA, 20 bp, Artificial sequence
  Primer
aaccaccagatcagccatgg

SEQ ID NO: 159 - DNA, 20 bp, Artificial sequence
  Primer
tgtgggtctgtgtgttcacc

SEQ ID NO: 160 - DNA, 20 bp, Artificial sequence
  Primer
tgaatagttcggcggtggag

SEQ ID NO: 161 - DNA, 20 bp, Artificial sequence
  Primer
gtgggtctgtgtgttcacca

SEQ ID NO: 162 - DNA, 20 bp, Artificial sequence
  Primer
tgaatagttcggcggtggag

(19) United States
(12) Patent Application Publication
(10) Pub. No.: US 2024/0122137 A1
(43) Pub. Date: Apr. 18, 2024

(54) QUANTITATIVE TRAIT LOCI (QTLS) ASSOCIATED WITH A HIGH-VARIN TRAIT IN CANNABIS

(71) Applicant: Puregene AG, Zeiningen (CH)

(72) Inventors: Claudio CROPANO, Zeiningen (CH); Dániel Árpád CARRERA, Zeiningen (CH); Gavin Mager GEORGE, Zeiningen (CH); Leron KATSIR, Zeiningen (CH); Maximilian Moritz VOGT, Zeiningen (CH); Michael Eduard RUCKLE; Yannik SCHLUP, Zeiningen (CH)

(21) Appl. No.: 18/278,370

(22) PCT Filed: Feb. 23, 2022
(86) PCT No.: PCT/IB2022/051583

§ 371 Date: Aug. 22, 2023

(30) Foreign Application Priority Data
Feb. 23, 2021 (GB) 2102532.5
Jan. 7, 2022 (GB) 2200183.8

(51) Int. Cl. A01H 1/04; C12Q 1/6895

(52) U.S. Cl. CPC A01H 1/045; C12Q 1/6895; C12Q 2600/13; C12Q 2600/156

(57) ABSTRACT

The present invention relates to methods of identifying Cannabis sativa plants comprising quantitative trait loci (QTLs) associated with a high-varin trait, and to Cannabis sativa plants comprising these QTLs. The invention also relates to plants with increased levels of varin content which are identified by the methods of the invention. The invention further relates to marker assisted selection and marker assisted breeding methods for obtaining plants having a high-varin trait, as well as to methods of producing Cannabis sativa plants with increased levels of varins and plants produced by these methods.

### BACKGROUND OF THE INVENTION

The present invention describes methods of identifying a Cannabis sativa plant comprising quantitative trait loci (QTLs) associated with a high-varin trait, and to Cannabis sativa plants comprising the QTLs. The invention also relates to plants with increased levels of varin content identified by the methods. The invention further relates to marker assisted selection and marker assisted breeding methods for obtaining plants having a high-varin trait, as well as to methods of producing Cannabis sativa plants with increased levels of varins and plants produced by these methods.

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are used for different purposes. Hemp is primarily used for industrial purposes, for example in feed, food, seed, fiber, and oil production. Conversely, HRT Cannabis is largely cultivated and bred for high concentrations of the pharmacological constituents, cannabinoids, derived from resin in the trichomes. Biomass, including the leaf and stem, of Cannabis can also be an important source of cannabinoids. However, there is recent interest from industrial producers in valuable, novel varieties based on the convergence of these two types.

Cannabis is the only species in the plant kingdom to produce phytocannabinoids. Phytocannabinoids are a class of terpenoid acting as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

The mechanism by which Cannabigerolic acid (CBGA) is synthesized was proposed by Lou et al (2019) (FIG. 1), based on in situ reconstitution of the cannabinoid pathway in yeast, but has not been demonstrated with in vitro enzyme assays or in vivo in Cannabis sativa tissues, and few of the genes encoding the enzymes in this pathway have been identified. The starting polyketide is hexanoic acid—a breakdown product of fatty acid metabolism—containing a C5 alkyl sidechain. Hexanoic acid is converted into an activated thioester, hexanoyl-CoA, in a reaction catalyzed by an unidentified acyl activating enzyme 1 (AAE1). In the Olivetolic Acid Cannabinoid Biosynthetic Pathway (OACB Pathway) (FIG. 1) Hexanoyl-CoA is subsequently lengthened with a malonyl-CoA by olivetol synthase (OLS) (a polyketide synthase (PKS)) followed by a cyclization step by olivetolic acid cyclase (OAC) to produce olivetolic acid (OA). Geranyl pyrophosphate (GPP) from the MEP pathway, together with CBGAS (a prenyltransferase 4 (PT4)) then prenylates OA to form C21 CBGA. Some of the enzymes in this pathway, however, are proposed to be promiscuous by using alternative short-chain fatty acid-CoAs (C1-C4) e.g., butanoyl-CoA as the starting molecule. In this way the same enzymes of the pathway may concomitantly produce divarinic acid, which is then prenylated to form C19 CBGVA, the precursor to the “varin” cannabinoids in a parallel pathway—the Divarinic Acid Cannabinoid Biosynthetic Pathway (DACB pathway) (FIG. 1). OLS have been shown to preferentially use hexanoyl-CoA as a substrate and C21 cannabinoids are usually present in higher quantities than their C19 analogs in Cannabis. It is, however, possible that an increasing percentage of C19:C21 cannabinoid can be achieved by a PKS showing higher affinity for C3 fatty acid-CoAs; or that higher substrate availability of butanoyl-CoA compared to hexanoyl-CoA may drive the reaction towards that of varin production (Gulck et a12020, Luo et a12019, Taura et a12009 and de Meijer and Hammond 2016). Changes in the percentage of varin compounds (THCV; THCVA; CBDV; CBDVA; CBGV; CBGVA; CBCV; CBCVA) to non-varin compounds (THCA; THC; CBDA; CBD; CBG; CBGA; CBC; CBCA) is indicative of an increase in the metabolic flux through the DACB-Pathway (FIG. 1). Furthermore, it cannot be excluded that an entirely undiscovered control mechanism exists.

CBDVA, THCVA and CBCVA are initially present in the plant as carboxylated acids that are decarboxylated down to their non-acidic forms CBDV, THCV and CBCV as a result of heating, aging or drying. CBDV, in particular, has received significant attention in the pharmaceutical Cannabis space. Clinical studies have shown its effectiveness as an anti-epileptic and anti-convulsant drug (Amada et al 2013) and it is being developed by GW Pharmaceuticals as a scheduled anti-epileptic drug. THCV is a neutral antagonist of the CB1 receptors and partial agonist of CB2 receptors. Although it is a homologue of THC, it does not present with psychoactive properties. In mice models it was shown to have counter-obesity effects through numerous metabolic processes by acting as an appetite suppressant, and by restoring insulin sensitivity in type-2 diabetic patients (Wargent et al 2013). It has also shown potential in the treatment for pain and inflammation (Bolognini et a12010) and in Parkinson's disease (Garcia et al 2011).

The utility of THCV and CBDV containing pharmaceuticals is currently hampered by incredibly low concentrations in known varieties. The present invention aims to provide Cannabis varieties and methods for obtaining Cannabis varieties with significantly higher concentrations of these valuable cannabinoids.

### SUMMARY OF THE INVENTION

The present invention relates to a method for identifying a Cannabis sativa plant comprising in its genome one or more QTLs for a high varin trait. The invention further relates to methods of producing a Cannabis sativa plant comprising in its genome a high-varin QTL identified by the method, or a high-varin trait associated with said high-varin QTL. In addition, the present invention relates to Cannabis sativa plants identified or produced according to the methods disclosed and to plant extracts obtainable from such Cannabis sativa plants. The invention also relates to Cannabis sativa plants containing a high-varin QTL or displaying the high-varin trait and to extracts thereof, including for use in methods of treatment. Also provided are quantitative trait loci and genes that control a high-varin trait in Cannabis sativa.

According to a first aspect of the invention there is provided for a method for identifying a Cannabis sativa plant comprising in its genome one or more high-varin QTLs, the method comprising the steps of: (i) providing a population of Cannabis plants; (ii) genotyping at least one plant from the population by detecting an allele of one or more polymorphisms associated with a high-varin trait as defined in Table 1 or Table 2; and (iii) identifying one or more plants containing the high-varin QTL.

In a first embodiment of the method of identifying a Cannabis sativa plant, the polymorphisms in Table 1 define a first high-varin QTL associated with the high-varin trait in the Cannabis sativa plant and the polymorphisms in Table 2 define a second high-varin QTL associated with the high-varin trait in the Cannabis sativa plant. In some embodiments, the plants identified by the method contain either the first high-varin QTL or the second high-varin QTL, or both the first high-varin QTL and the second high-varin QTL.

In one embodiment of the method of identifying a Cannabis sativa plant, the population of Cannabis plants may be obtained by crossing at least one donor parent plant having in its genome one or more of the high-varin QTLs with at least one recipient parent plant that does not have one or more of the high-varin QTLs in its genome. Preferably, the donor parent plant displays a high-varin trait. For example, the donor parent plant may have a total varin (C19) cannabinoid content of about 10% of a total C21 cannabinoid content in the same plant tissue as measured by UPLC. Most preferably, the donor parent plant may have a varin (C19) cannabinoid content in the plant tissue that is approximately equal to or greater than the non-varin (C21) cannabinoid content in the same plant tissue as measured by UPLC.

In a further embodiment of the method of identifying a Cannabis sativa plant, the genotyping is performed by PCR-based detection using molecular markers, sequencing of PCR products containing the one or more polymorphisms, targeted resequencing, whole genome sequencing, or restriction-based methods, for detecting the one or more polymorphisms. The molecular markers used to genotype the plant may be the KASP molecular markers provided in Table 3. In another embodiment, the region of interest containing the QTL may be sequenced using the primers provided in Table 5 or 6.

In an embodiment of the method of identifying a Cannabis sativa plant, the molecular markers may be for detecting polymorphisms at regular intervals within each, or both, of the QTLs such that recombination can be excluded.

In an alternative embodiment of the method of identifying a Cannabis sativa plant, the molecular markers may be for detecting polymorphisms at regular intervals within each, or both, of the QTLs such that recombination can be quantified to estimate linkage disequilibrium between a particular polymorphism and a high-varin phenotype conferred by the one or more high-varin QTLs.

According to a second aspect of the present invention there is provided for a method of producing a Cannabis sativa plant comprising in its genome one or more high-varin QTLs, the method comprising the steps of: (i) providing a donor parent plant having in its genome a high-varin QTL characterized by an allele of one or more polymorphisms associated with a high-varin trait as defined in Table 1 or Table 2; (ii) crossing the donor parent plant having the high-varin QTL with at least one recipient parent plant that does not have the high-varin QTL to obtain a progeny population of Cannabis plants; (iii) screening the progeny population of Cannabis plants for the presence of the high-varin QTL; and (iv) selecting one or more progeny plants having the high-varin QTL.

In one embodiment of the method of producing a Cannabis sativa plant, the method may further comprise the steps of: (v) crossing the one or more progeny plants with the donor recipient plant; or (vi) selfing the one or more progeny plants.

According to one embodiment of the method of producing a Cannabis sativa plant, the polymorphisms in Table 1 define a first high-varin QTL associated with the high-varin trait in the Cannabis sativa plant and the polymorphisms in Table 2 define a second high-varin QTL associated with the high-varin trait in the Cannabis sativa plant. In some embodiments, the progeny plants may contain either the first high-varin QTL or the second high-varin QTL, or both the first high-varin QTL and the second high-varin QTL.

In a further embodiment of the method of producing a Cannabis sativa plant, the one or more progeny plants having the one or more high-varin QTLs display a high-varin trait. For example, the one or more progeny plants having the high-varin QTL may have a total varin (C19) cannabinoid content of about 10% of a total C21 cannabinoid content in the same plant tissue, as measured by UPLC. Most preferably, the one or more progeny plants having the high-varin QTL may have a varin (C19) cannabinoid content in the plant tissue that is approximately equal to or greater than the C21 cannabinoid content in the same plant tissue as measured by UPLC.

In one embodiment of the method of producing a Cannabis sativa plant, the screening may comprise genotyping at least one plant from the progeny population with respect to the high-varin QTL by detecting the allele of the one or more polymorphisms associated with the high-varin trait as defined in Table 1 or Table 2. Numerous methods of genotyping are known in the art. For example, the genotyping may be performed by PCR-based detection using molecular markers, sequencing of PCR products containing the one or more polymorphisms, targeted resequencing, whole genome sequencing, or restriction-based methods, for detecting the one or more polymorphisms.

In an embodiment of the method of producing a Cannabis sativa plant, the molecular markers may be for detecting polymorphisms at regular intervals within each, or both, of the QTLs such that recombination can be excluded.

According to a further embodiment of the method of producing a Cannabis sativa plant, the recipient parent plant may have one or more desirable characteristics unrelated to varin content and the one or more progeny plants having a high-varin QTL may have the one or more desirable characteristics unrelated to varin content.

According to a third aspect of the present invention there is provided for a method of producing a Cannabis sativa plant comprising a high-varin trait, the method comprising introducing a high-varin QTL characterized by an allele of one or more polymorphisms associated with the high-varin trait as defined in Table 1 or Table 2 into a Cannabis sativa plant.

In some embodiments, the polymorphisms in Table 1 define a first high-varin QTL associated with the high-varin trait in the Cannabis sativa plant and the polymorphisms in Table 2 define a second high-varin QTL associated with the high-varin trait in the Cannabis sativa plant. In one embodiment, a plant comprising one or both of the high-varin QTLs has increased varin (C19) cannabinoid content compared to a plant that does not comprise the high-varin QTL. In some embodiments, the plants produced by the method may contain either the first high-varin QTL or the second high-varin QTL, or both the first high-varin QTL and the second high-varin QTL

In one embodiment of the method of this aspect of the invention, the plant comprising the high-varin QTL has increased varin (C19) cannabinoid content in the plant tissue thereof compared to a plant that does not comprise the high-varin QTL. For example, the plant may have a total varin (C19) cannabinoid content of about 10% of a total C21 cannabinoid content in the same plant tissue as measured by UPLC. Most preferably, the plant may have a varin (C19) cannabinoid content in the plant tissue that is approximately equal to or greater than the C21 cannabinoid content in the same plant tissue as measured by UPLC.

In an embodiment of the method of this aspect of the invention, introducing a high-varin QTL may comprise crossing a donor parent plant in which the high-varin QTL is present, with a recipient parent plant in which the high-varin QTL is not present.

In an alternative embodiment of the method of this aspect of the invention, introducing a high-varin QTL may comprise genetically modifying the Cannabis sativa plant. Numerous methods of genetically modifying a plant are known in the art. For example, an allele of one or more of the polymorphisms associated with the high-varin trait as defined in Table 1 or Table 2 may be introduced into a plant by mutagenesis and/or gene editing. In particular, the methods of genetically modifying a plant may be selected from the group consisting of CRISPR-Cas9 targeted gene editing, heterologous gene expression using various expression cassettes; TILLING, and non-targeted chemical mutagenesis using e.g. EMS. Alternatively, the QTLs associated with the high-varin trait characterized by an allele of one or more of the polymorphisms associated with the high-varin trait as defined in Table 1 or Table 2, or a part thereof, may be introduced into a plant by transformation of the plant with a vector comprising a gene cassette including one or both of the QTLs defined herein.

According to a fourth aspect of the present invention there is provided for a Cannabis sativa plant identified according to the methods described in the first aspect herein, or produced according to the second or third aspects herein, provided that the plant is not exclusively obtained by means of an essentially biological process.

According to a fifth aspect of the present invention, there is provided for a Cannabis sativa plant comprising a high-varin QTL characterized by an allele of one or more polymorphisms associated with a high-varin trait as defined in Table 1 or Table 2, provided that the plant is not exclusively obtained by means of an essentially biological process. For example, the plant may comprise a first high-varin QTL associated with the high-varin trait in the Cannabis sativa plant characterized by an allele of one or more polymorphisms associated with a high-varin trait as defined in Table 1 and a second high-varin QTL associated with the high-varin trait in the Cannabis sativa plant characterized by an allele of one or more polymorphisms associated with a high-varin trait as defined in Table 2.

In one embodiment of the plant of the present invention, the plant may have an increased varin (C19) cannabinoid content in the plant tissue thereof compared to a plant that does not comprise the high-varin QTL. For example, the plant may have a total varin (C19) cannabinoid content of about 10% of a total non-varin (C21) cannabinoid content in the same plant tissue as measured by UPLC. Most preferably, the plant may have a varin (C19) cannabinoid content in plant tissue that is approximately equal to or greater than the non-varin (C21) cannabinoid content in the same plant tissue as measured by UPLC.

According to a further aspect of the present invention there is provided for a plant extract obtainable from a Cannabis sativa plant as described herein. Preferably, the plant extract has an increased varin (C19) cannabinoid content in the plant tissue thereof compared to a plant that does not comprise the high-varin QTL. For example, the plant extract may have a total varin (C19) cannabinoid content of about 10% of a total C21 cannabinoid content, such as a varin (C19) cannabinoid content in the plant tissue that is approximately equal to or greater than the C21 cannabinoid content in the same plant tissue as measured by UPLC.

According to yet another aspect of the present invention there is provided for a Cannabis sativa plant or plant extract as described herein for use in a method of treatment of epilepsy, obesity, pain, inflammation, diabetes, and/or Parkinson's disease, or for use as an anti-convulsant and/or appetite suppressant, or for use in a method of restoring insulin sensitivity in diabetic patients. Methods of treatment of epilepsy, obesity, pain, inflammation, diabetes, and/or Parkinson's disease, or methods for use as an anti-convulsant and/or appetite suppressant, or method of restoring insulin sensitivity in diabetic patients.

According to a further aspect of the present invention, there is provided for a quantitative trait locus that controls a high-varin trait in a Cannabis sativa plant, wherein the quantitative trait locus has a sequence that corresponds to nucleotides 5139731 to 47648106 of NC_044373.1 and contains an allele of one or more polymorphisms associated with the high-varin trait as defined in Table 1.

In another aspect of the present invention there is provided for a quantitative trait locus that controls a high-varin trait in a Cannabis sativa plant, wherein the quantitative trait locus has a sequence that corresponds to nucleotides 68296752 to 70024415 of NC_044378.1 and contains an allele of one or more polymorphisms associated with the high varin trait as defined in Table 2.

In a further aspect of the present invention there is provided for a gene that controls a high-varin trait in a Cannabis sativa plant, wherein the gene encodes a 4-coumarate--CoA ligase-like 1. Preferably, the gene corresponds to LOC115712547 with reference to the CS10 genome and encodes a 4-coumarate--CoA ligase-like 1.

According to another aspect of the present invention there is provided for a gene that controls a high-varin trait in a Cannabis sativa plant, wherein the gene encodes a GDSL lipase, an acyl-acyl carrier protein, or an oxysterol binding protein. Preferably, the gene is as defined in Table 7 with reference to the CS10 genome and encodes a GDSL lipase.

### BRIEF DESCRIPTION OF THE FIGURES

Non-limiting embodiments of the invention will now be described by way of example only and with reference to the following figures:

FIG. 1: Biosynthesis pathway for the C21 and C19 cannabinoids. The use of butanoly-CoA as an alternative substrate for OLS is proposed by Lou et al. 2019, based on in situ reconstitution of the cannabinoid pathway in yeast, but has not been demonstrated with in vitro enzyme assays or in vivo in Cannabis sativa tissues.

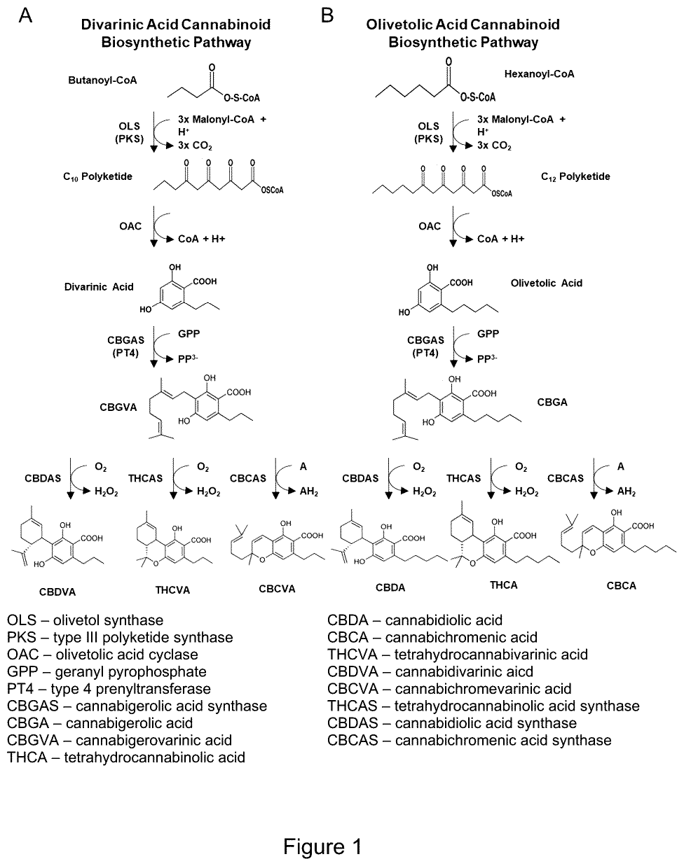

FIG. 2: Correlation of total varin (C19 cannabinoids)/total cannabinoids (C19+C21) derived from leaf and flower. Leaf total varin/total cannabinoids is plotted against flower total varin/total cannabinoids. Linear regression quantifies the strength of the correlation.

### SEQUENCES

The nucleic acid and amino acid sequences listed herein and in any accompanying sequence listing are shown using standard letter abbreviations for nucleotide bases, and the standard one or three letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

Molecular analytic tools can be used to breed Cannabis varieties, including for commercial and research use. Genomic regions controlling the production of cannabinoids, such as the production of varins can be identified using these tools. Genetic or molecular markers to these regions can be used in Cannabis breeding to identify plants with a desired phenotype, such as high-varin content. Methods and compositions for providing a plant with a desirable cannabinoid profile are provided, along with related compositions and plants.

Methods are provided herein for identifying and obtaining plants with a high-varin trait containing elevated varinic cannabinoid content. The inventors of the present invention have made use of genome-wide association studies (GWAS) of input Cannabis varieties, to determine genomic regions and/or polymorphisms that statistically associated with the high-varin trait in Cannabis plant material. In one embodiment of the invention, these polymorphisms may be used for marker assisted selection (MAS) of plants containing the high-varin trait. In one embodiment of the present invention, Quantitative Trait Loci (QTL) associated with the high-varin trait were identified in Cannabis sativa. Tables 1 and 2 herein provide a number of polymorphisms which define a QTL associated with the high-varin trait, termed qtIV1 found on Chromosome 4 (NC_044373.1) and qtIV2 found on Chromosome 7 (NC_044378.1). In some embodiments one or more of the identified SNPs can be used to incorporate the high-varin trait from a donor plant, containing the QTL associated with the high-varin trait, into a recipient plant. For example, the incorporation of the high-varin phenotype may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying one or more QTLs that are characterized by a haplotype comprising of a series of polymorphisms in linkage disequilibrium. The QTLs each display limited frequency of recombination within the QTLs. Preferably the polymorphisms are selected from Tables 1 and/or 2 herein, representing qtIV1 and qtIV2, respectively. Molecular markers may be designed for use in detecting the presence of the polymorphisms and thus the QTLs. Further, the identified QTL polymorphisms and/or associated molecular markers may be used in a Cannabis breeding program to predict the high-varin chemotype of plants in a breeding population and can be used to produce Cannabis plants in which CBGVA (and/or CBDVA and CBCVA and THCVA) is increased compared to plants of a control population in which the QTL is not present. In plants generated from such a breeding population, the profile of various C21 cannabinoids (including THCA, CBDA, CBCA, or CBGA) will be determined by the active synthases inherited from each parent and selected in the offspring. The QTLs described herein will directly alter the inherent ability of the plant to produce these cannabinoids. The introduction of the qtIV1 or qtIV2 will, however, determine the percent total C19 (including but not limited to CBGVA and CBDVA and CBCVA and THCVA) to total C21 in plant tissue. For example, in some embodiments, the varin levels can be increased in a progeny plant relative to a recipient parent plant by crossing the recipient parent plant with a donor parent plant. In particular, the total varin levels may be increased such that the progeny contains about 10%, or 50%, or 100%, or greater, total C19 cannabinoids compared to C21 cannabinoids where the recipient parent plant contains a percentage of C19 cannabinoids as a proportion of the total cannabinoid content that is less than the donor plant. In another embodiment, a crossing of a donor plant to a recipient plant may result in at least a 10 increase in the C19 cannabinoid content of offspring compared to a recipient parent plant. In one embodiment, the high-varin trait is defined as a trait that increases the C19-cannabinoid content of the progeny of a recipient plant relative to the recipient plant's C19-cannabinoid content. Plants expressing the high-varin trait may have more than 1% C19 cannabinoids, which is relative to Cannabis sativa plants that do not have the high-varin trait and contain less than 1% C19 cannabinoids.

As used herein, reference to a plant or a variety with “high-varin” or “high-varin trait” refers to a plant or a variety that has a varin (C19) cannabinoid content in the mature flower or leaf tissue that is >10% total C19 cannabinoids when compared to the total C21 cannabinoids in the same flower or leaf tissue as measured by UPLC. Preferably C19 cannabinoid content is equal to or greater than the C21 cannabinoid content in the same mature flower or leaf tissue as measured by UPLC.

As used herein a “quantitative trait locus” or “QTL” is a polymorphic genetic locus with at least two alleles that differentially affect the expression of a continuously varying phenotypic trait when present in a plant or organism, or a part thereof, which is characterised by a series of polymorphisms in linkage disequilibrium with each other.

As used herein, the term “high-varin QTL” or “high-varin quantitative trait locus” refers to a quantitative trait locus comprising part or all of the qtIV1, which is characterized by one or more of the polymorphisms described in Table 1 or a quantitative trait locus comprising part or all of the qtIV2, which is characterized by one or more of the polymorphisms described in Table 2, or which comprises part or all of both quantitative trait loci qtIV1 and qtIV2.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium have the tendency to be caused by genetic linkage due their location on the same chromosome.

As used herein, the term “high-varin haplotype” refers to the subset of the polymorphisms contained within a high-varin QTL which exist on a single haploid genome complement of the diploid genome, and which are in linkage disequilibrium with the high-varin trait.

As used herein, the term “donor parent plant” refers to a plant that is either homozygous or heterozygous for the high-varin haplotype or which contains a high-varin QTL identified herein.

As used herein, the term “recipient parent plant” refers to a plant that is not heterozygous or homozygous for containing the high-varin QTL, qtIV1, or the high-varin QTL, qtIV2, or the high-varin haplotype but which may contain varin that is induced through the action of a discreet genomic region other than that defined by qtIV1 and/or qtIV2.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “high-varin allele” refers to the haplotype allele within a particular QTL that confers, or contributes to, high-varin phenotype, or alternatively, is an allele that allows the identification of plants with high-varin phenotype that can be included in a breeding program (“marker assisted breeding” or “marker assisted selection”) and which is defined in Table 1 and/or Table 2 herein with an asterisk.

The term “nucleic acid” encompasses both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

The term “isolated”, as used herein means having been removed from its natural environment.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially reduce the antigenicity of the expressed fusion protein or of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2× SSC), and a second wash for 10 min at 65° C. in Wash Buffer B (0.1% SDS; 0.5% SSC).

### Methods of Identifying a QTL or Haplotype Responsible for High-Varin Phenotype and Molecular Markers Therefor

In some embodiments, methods are provided for identifying a QTL or haplotype responsible for high varin content and for selecting plants with the high-varin trait. In some embodiments, the methods may comprise some or all of the steps of:

a. Identifying a plant that contains a high-varin cannabinoid content within a breeding program.

b. Establishing a population by crossing the identified plant to itself (selfing) or a recipient parent plant.

c. Genotyping the resultant F1, or subsequent populations, for example by sequencing methods.

d. Performing association studies, such as genome-wide association studies, including phenotyping and linkage analysis, to discover QTLs and/or polymorphisms contained within the QTL.

e. Optionally, identifying Cannabis paralogs of previously characterized genes that may be involved in the production of divarinic acids.

f. Developing molecular markers that detect one or more polymorphisms linked to QTLs, alleles within these QTLs, or existing or induced polymorphisms.

g. Validating the molecular markers by determining the linkage disequilibrium between the marker and the high-varin trait

### Trait Development and Introgression

In some embodiments, methods are provided for marker assisted breeding (MAB) or marker assisted selection (MAS) of plants having a high-varin trait. The methods may comprise some or all of the steps of:

a. Identifying a plant that contains a high-varin cannabinoid content.

b. Establishing a population by crossing the identified plant to itself (selfing) or another recipient parent plant.

c. Genotyping and phenotyping the resultant F1, or subsequent, populations, for example by sequencing methods and cannabinoid quantification by UPLC methods.

d. Performing association studies, such as genome-wide association studies, inputting phenotype and genotype information to identify genomic regions enriched with polymorphisms associated with the high-varin trait, to discover QTLs and/or polymorphisms contained within the QTL.

e. Optionally, identifying Cannabis paralogs of previously characterized genes that may be involved in the production of divarinic acids.

f. Developing molecular markers that detect one or more polymorphisms linked to QTLs, alleles within these QTLs, or existing or induced polymorphisms.

9. Using the molecular markers when introgressing the QTLs or polymorphisms into new or existing Cannabis varieties to select plants containing the high-varin haplotype or the high-varin trait.

### QTLs and Marker Assisted Breeding

In some embodiments, during the breeding process, selection of high-varin plants may be based on molecular markers designed to detect polymorphisms linked to genomic regions that control the trait of interest by either an identified or an unidentified mechanism. Unidentified genetic mechanisms may, for example, have a direct or pleiotropic effect on varin accumulation in a plant. Examples include genes controlling trichome or organ development, metabolite transport, general regulators of transcription and translation, enzymes that affect varinic acid biosynthetic pathway or other cannabinoids, or other pleiotropic factors. In some embodiments, QTLs containing such elements are identified using association studies, including genome-wide association studies. Knowledge of the mode-of-action is not required for the functional use of these genomic regions in a breeding program. Identification of regions controlling unidentified mechanisms may be useful in obtaining plants with elevated varin cannabinoid content, based on identification of polymorphisms that are either linked to, or found within QTLs that: (i) are associated with the high-varin phenotype using AS; (ii) affect the expression or activity of genes encoding enzymes that produce precursors to CBGVA; and/or (iii) act to increase the percent total C19 to C21 cannabinoid through an unidentified mechanism.

In some embodiments, QTLs with unidentified or non-obvious modes of action, including pleiotropic effects on varin cannabinoid biosynthesis include: (i) QTLs that contain genes required for protein complex formation of enzymes upstream of CBGVA; (ii) QTLs that contain genes encoding protein which interact with one or more of the upstream or downstream enzymes around CBGV or CBGVA and alter their activity; (iii) QTLs that contain genes encoding proteins that inhibit the activity, transcription or translation of enzymes and genes related to the production of acidic (C21) cannabinoid biosynthesis; (iv) QTLs that promote transcription, translation or activity of enzymes and genes related to the production of varin (C19) cannabinoid biosynthesis; and/or (v) QTLs that promote the production of THCVA, CBDVA, or CBCVA rather than THCA, CBDA and CBCA.

### Construction of Breeding Populations

Breeding populations are the offspring of sexual reproduction events between two or more parents. The parent plants (FO) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the high-varin trait are identified within synthetic populations of plants capable of revealing dominant, recessive, or complex traits. In one embodiment of the invention, a genetically diverse population of Cannabis varieties, that are used to produce the synthetic population can be integrated into a breeding program of unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the following methods are employed to integrate the plants into a breeding program include some or all of the following:

a. Growing plants in rich media or soils under artificial lighting;

b. Cloning of plants, often through a multitude of sub-cloning cycles;

c. Introduction of plants into in vitro, sterile growth environments, and subsequent removal to standard growth conditions;

d. Exposure to mutagens such as EMS, colchicine, silver nitrate, ethidium bromide, dinitroanalines, high concentrations mono or poly-chromatic light sources;

e. Growing plants under highly stressful conditions which include restricted space, drought, pathogen, atypical temperatures, and nutrient stresses;

### High-Varin Trait Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a trait of interest, such as the high-varin trait, may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype. In this case the trait is the varin content or percent total C19 to C21 cannabinoids. In a population generated by crossing, the amount of linkage disequilibrium (LD) is reduced between genetic marker and the QTL as a function of genetic distance in Cannabis varieties with similar genome structures. Simple association mapping is performed by biparental crosses of two closely related lines where one line has a phenotype of interest and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, varin content of the plants as determined using leaf cannabinoid assays. Using the association studies described herein, together with accurate phenotyping, this method is able to identify genomic regions, QTLs and even specific genes or polymorphisms responsible for elevated varinic acid content that are directly introduced into recipient lines. Polygenic phenotypes may also be identified using the methods described herein.

In one embodiment, the structured population is grown to the flowering stage. To characterize the phenotypes of the lines they are clonally reproduced so the phenotypic data can be collected in feasible replicates. Parts of the plant including, but not limited to, the inflorescence, leaves, and trichomes, are harvested and analyzed for their varinic cannabinoid content by high-pressure liquid chromatography (HPLC) or ultra performance liquid chromatography (UPLC) linked to a detector. Where available the chromatogram peaks corresponding to varinic cannabinoids are identified by comparison to purified standards. If no standards are available, the cannabinoids can be identified by their mass fragmentation on the mass spectrometer, or fractions can be collected and identified by other means.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Markers detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system and may be PCR primers, such as those described in the examples below. For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a Cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals, and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from Cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two or more of the SNPs in the haplotype can be used to identify the presence of the QTL and by association, the high-varin phenotype.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the high-varin trait of the offspring.

According to some embodiments, any polymorphism in linkage disequilibrium with the high-varin QTL can be used to determine the presence of the haplotype in a breeding population of plants, as long as the polymorphism is unique to the high-varin trait in the donor parent plant when compared to the recipient parent plant.

In some embodiments of the invention, the donor parent plant is a plant that has been genetically modified to include a high-varin QTL defined by a polymorphism, for example any or all of the polymorphisms of Table 1 or Table 2.

In some embodiments, donor parent plants, as described above, are used as one of two parents to create breeding populations (F1) through sexual reproduction. Methods for reproduction that are known in the art may be used. The donor parent plant provides the trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a high-varin QTL allele or high-varin haplotype.

In some embodiments, the presence of the high-varin allele or high-varin haplotype in plants to be used in the F1 cross is determined using the described molecular markers. In some embodiments, the resulting F2 progeny is/are screened for any of the high-varin polymorphisms described herein.

The plants at any generation can be produced by asexual means like cutting and cloning, or any method that yields a genetically identical offspring.

Production of High-Varin Cannabis sativa

In some embodiments, a Cannabis sativa plant may be converted into a high-varin plant according to the methods of the present invention by providing a breeding population where the donor parent plant contains the high-varin QTL associated with the high-varin trait and recipient parent plant contains relatively low varin in comparison.

In some embodiments, the recipient parent plant used in the creation of the breeding population does not contain the high-varin QTL or haplotype. In some embodiments the recipient parent plant contains less than 10% varin (C19) cannabinoids compared to the C21 cannabinoid content in the dry mass of mature inflorescence.

In some embodiments the high-varin phenotype may be introduced into a recipient parent plant by crossing it with a donor parent plant comprising a high-varin phenotype. In some embodiments the donor parent plant comprising a high-varin phenotype comprises one or both of qtIV1 and qtIV2. In some embodiments the donor parent plant comprising a high-varin phenotype and a contiguous genomic sequence characterized by one or more of the polymorphisms of Table 1 or Table 2. In some embodiments, the donor parent plant is any Cannabis variety that is cross fertile with the recipient parent plant.

In some embodiments, MAS or MAB may be used in a method of backcrossing plants carrying the high-varin trait to a recipient parent plant. For example, an F1 plant from a breeding population can be crossed again to the recipient parent plant. In some embodiments, this method is repeated.

In some embodiments, the resulting plant population is then screened for the high-varin trait using MAS with molecular markers to identify progeny plants that contain one or more high-varin polymorphisms, such as those described in Table 2, indicating the presence of an allele of the QTL associated with a high-varin phenotype. In another embodiment, the population of Cannabis plants may be screened by measuring cannabinoids directly or by other analytical methods known in the art to identify plants with desired characteristics.

### Methods to Genetically Engineer Plants to Achieve High-Varin Using Mutagenesis or Gene Editing Techniques

Identifying QTLs, and individual polymorphisms, that correlate with a trait when measured in an F1, F2, or similar, breeding population indicates the presence of one or more causative polymorphisms in close proximity the polymorphism detected by the molecular marker. In some embodiments, the polymorphisms associated with the high-varin trait is introduced into a plant by other means so that a trait, such as the high-varin trait, can be introduced into plants that would not otherwise contain associated causative polymorphisms.

The entire QTLs of parts thereof which confer the varin trait described herein may be introduced into the genome of a Cannabis plant to obtain plants with a high-varin phenotype through a process of genetic modification known in the art, for example, but not limited to, heterologous gene expression using various expression cassettes.

The trait described herein may be introduced into the genome of a Cannabis plant to obtain plants that include the causative polymorphisms and the potential to display a high-varin phenotype through processes of genetic modification known in the art, for example, but not limited to, CRISPR-Cas9 targeted gene editing, TILLING, non-targeted chemical mutagenesis using e.g. EMS.

Plants may be screened with molecular markers as described herein to identify transgenic individuals with a high-varin QTL or polymorphism, following the genetic modification.

In some embodiments, Cannabis plants comprising one or both of qtIV1 and qtIV2, or comprising one or more of the polymorphisms of Table 1 or Table 2 associated with qtIV1 or qtIV2, respectively are provided. In some embodiments the qtIV1 and/or qtIV2, or one or more polymorphisms associated therewith are introduced into the plants. For example, by genetic engineering. In some embodiments the one or more polymorphisms are introduced into the plants by breeding, such as by MAS or MAB, for example as described herein.

Accordingly, in a further embodiment, Cannabis sativa plants comprising one or both of qtIV1 and qtIV2, or one or more polymorphisms associated therewith, are provided, with the proviso that the plant is not exclusively obtained by means of an essentially biological process.

The invention also relates to a plant extract obtainable from a Cannabis sativa plant provided herein. It is preferred that the plant extract has a C19 cannabinoid content that is equal to or greater than the C21 cannabinoid content as measured in the same mature flower.

Methods of Use of the Plant, Parts Thereof and/or Extracts Thereof of the Invention

In further embodiments, the invention relates to the plant extract of a plant or plant part provided herein for use in the treatment of epilepsy, obesity, pain, inflammation, diabetes, and/or Parkinson's disease, or for use as an anti-convulsant and/or appetite suppressant, or for use in restoring insulin sensitivity in diabetic patients. Also provided are methods of treatment of epilepsy, obesity, pain, inflammation, diabetes, and/or Parkinson's disease, or methods of preventing or treating convulsions, or methods of suppressing appetite, or methods of restoring insulin sensitivity in diabetic patients using the plant extracts. In further embodiments, the plant extract is provided for non-medical use, for example recreational use.

Provided herein are also products containing the plant, parts thereof and/or extracts thereof. For example, provided herein is a Cannabis cigarette or components of a smokable product containing parts of the plants provided herein.

The following examples are offered by way of illustration and not by way of limitation.

### EXAMPLE 1

### Plant Growth and Cannabinoid Analysis

The inventors of the present invention have identified two QTLs associated with a high-varin trait as detailed in UK patent application No. 2102532.5 and UK patent application No. 2200183.8, which are incorporated by reference herein in their entirety.

To quantify the chemotypic diversity of the collection with respect to varin production, 94 plants were grown on an outdoor field in Zeiningen, Switzerland over the summer of 2019. The plants flowered naturally under shortening days. Flowers were harvested from the primary flowering stem in mid-October, dried, and analyzed for their constituent cannabinoid content. Among the population of plants grown outdoors, 20 000 110 0000 was identified as having the highest proportion of CBDVA to CBDA. The presence of CBDVA was extremely rare with only two of the plants in the population having relative CBDVA to CBDA proportions more than 10% and they share a parent. Therefore, it is likely that this parent carries a novel genetic element responsible for C19 cannabinoid production. Only in one plant, 20 000 110 0000, did CBDVA accumulate to greater than 30%. In absolute values 20 000 110 0000 produces approximately 9% CBDA and 3% CBDVA with few other cannabinoids. 20 000 110 0000 was self-fertilized to create the 20 000 110 0000 S1 population. This was done between two clones of the plant previously identified. Through a sex reversal process, known in the art, the one clone was induced to produce pollen (pollen donor), which was used to fertilize the other clone (pollen recipient) in a controlled environment preventing outside pollen contamination.

Seeds of the 20 000 110 0000 S1 population were sown and grown in growth chambers for more than 24 days prior to sampling. Briefly, plants were grown in pots containing soil, in a chamber at room temperature with rapid air circulation. Plants were provided with approximately 600 μmol·m−2·s−1 of light provided by high-pressure sodium lamps in 18 h-day/6 h-night lighting regime. Cannabinoid assays were performed on 130 plants to determine the proportion of varin produced by each individual of the population according to the methods described below and the proportion of varin was calculated.

This data was used to create two subsets of the population of plants, presenting with a “low-varin” and “high-varin” proportion of C19:C21 cannabinoids. DNA was extracted from all of the plants in the 20 000 110 0000 S1 population using a commercial kit (Mag-Bind Plant DNA DS Kit from Omega Bio-tek) according to the manufacturer's instructions. Two pools of DNA were created using only the extracts from plants in a “low” subset, or a “high” subset consisting of 29 plants with low C19:C21 cannabinoid ratio and 48 plants with the highest proportion of C19:C21 cannabinoids based on LCA analysis, respectively. Both DNA pools were created using equimolar concentrations of each individual DNA extract.

Based on the identification of a QTL associated with a high-varin trait from the 20 000 110 0000 S1 population, the inventors undertook genome-wide analysis of a population of plants, GID:21 001 800 0000 known to be segregating for the high varin trait, in order to identify useful SNPs for identifying and obtaining plants with the high varin trait as described herein. This population was generated from a population derived from a selfing of GID: 20 000 110 0000 from the previous patent application selected for the high varin trait that were themselves selfed generating a population GID:20 004 091 0000. These were bulk crossed to a population derived from a distinct population of plants also segregating for the varin trait. The progeny of these crosses are GID: 21 001 800 0000.

To investigate the genetic basis of the high-varin phenotype in leaf and flower plant parts, seeds of population GID:21 001 800 known to be segregating for the high-varin trait were grown on an outdoor field in Zeiningen, Switzerland over the summer of 2020. To identify genomic regions and/or polymorphisms statistically associated with the high-varin trait, plant material from 86 individuals of GID:21 001 800 was harvested for genotyping and chemotyping. Leaf tissue was harvested mid-October for chemotyping as well as for DNA extraction. The plants flowered naturally under shortening days. Flowers were harvested from the primary flowering stem in mid-October, dried, and analyzed for their constituent cannabinoid content.

Cannabinoid assays (CAs) were performed to determine the correlation, if any, between flower and leaf cannabinoid content. CA analysis requires a small leaf tissue sample for rapid extraction in methanol and is a qualitative measure of cannabinoid content. Although leaf analyses detected compounds that aren't present in the mature flowers, the percent total CBDVA to CBDA is sufficiently consistent in these analyses to discriminate between varin-producing and non-varin-producing plants. CA analysis does not require flowering for chemotyping, and therefore allows for early-stage rapid discrimination between varin producers and varin non-producers. These data can be used for subsequent trait association studies.

Cannabinoid assays using leaf material were performed by adding 1000 μl pure methanol to a brown, light-excluding, 1.5 ml microcentrifuge tube. A leaflet from a mature leaf was placed immediately into the tube and incubated at room temperature for 5 min. Leaves were then removed from the tube with a pair of tweezers, and the tube containing the methanol extract was centrifuged for 10 min at maximum speed. Supernatant was filtered through a 0.2 μm microfilter into a new tube. Undiluted samples of 550 μl were measured by directly adding to the UPLC vial.

Cannabinoid extraction from flower material was performed through mechanical homogenization of ≈500 mg of plant flower material in the presence of 15 ml HPLC grade methanol (HiPerSolv CHROMANORM methanol, CAS:67-56-1) in disposable 50 ml test tubes. A 1m1 aliquot of the crude extract was clarified through centrifugation, the resulting supernatant was later filtered through a 0.2 μm PTFA syringe-filter and diluted as needed with methanol.

The cannabinoid assay was run on a 1290 Infinity II Agilent HPLC system equipped with DAD, temperature-controlled column compartment, multisampler, and quaternary pump. The separation of the analytes was achieved on a Kinetex 1.7 μm EVO C18 100A 100×1.2 mm column. Full spectra were recorded from 200 to 400 nm, and absorbance at 230 nm was used to quantify all analytes.

Instrument control, data acquisition, and integration were achieved with OpenLAB CDS (Agilent Technologies) software, applying an identification and quantification method based on an 8-level external standards calibration curve. To confirm the analyte identity in plant material, retention time and peak purity were compared with the signal acquired on certified reference materials (CRMs).

The calibration curve used for quantification was obtained by analyzing serial dilutions of an in house produced mixture containing 13 commercially available cannabinoids CRMs, namely Cannabidivarin (CBDV), Cannabidivarinic acid (CBDVA), Tetrahydrocannabivarin (THCV), Cannabidiol (CBD), Cannabigerol (CBG), Cannabidiolic acid (CBDA), Cannabinol (CBN), Cannabigerolic acid (CBGA), Delta-9-tetrahydrocannabinol (d9-THC), Delta-8-tetrahydrocannabinol (d8-THC), Cannabichromene (CBC), Tetrahydrocannabinolic acid (THCA), and Cannabichromenic acid (CBCA).

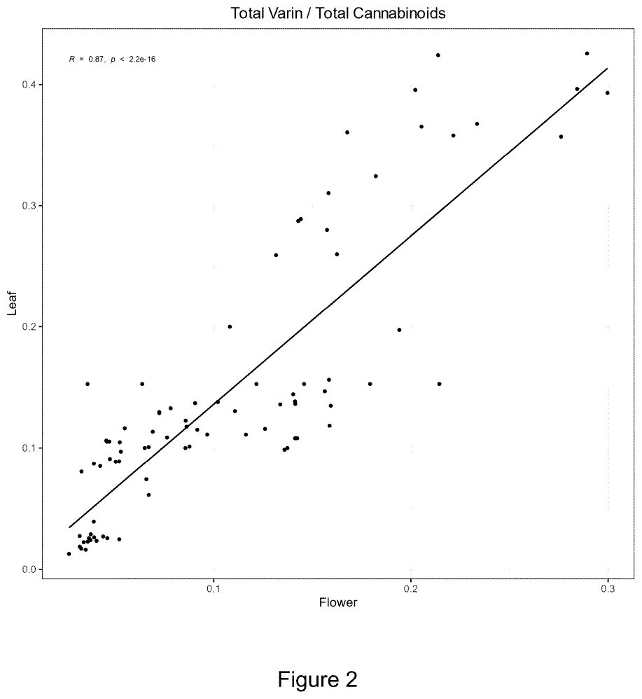

When evaluating the cannabinoid content in plants of population GID: 21 001 800 0000 the inventors found a strong correlation between the percent total varin cannabinoids to total cannabinoids between leaf and flower as shown in FIG. 2. This correlation indicates a common mechanism regulating the percent total varin cannabinoid to total cannabinoid in flowers and leaves. Increases in total cannabinoid content in plants, would increase the amount of varin cannabinoids but not result in an increased percent varin cannabinoids to total cannabinoids.

### EXAMPLE 2

### DNA Extraction, Marker Panel Identification and Genome-Wide Analysis (GWAS)

Genotype information was combined with phenotypes previously collected to perform GWAS analyses.

DNA was extracted from all of the plants using an adapted kit with “sbeadex” magnetic beads by LGC Genomics, which was automated on a KingFisher Flex with 96 Deep-Well Head by Thermo Fisher Scientific. Leaf discs about 70 mg were placed in Eppendorf tubes with porcelain beads and immersed in liquid N₂ and then homogenized with Star Beater from VWR at a frequency of 1/25 for 2 minutes. 400 μl Lysis buffer PVP, supplemented with 5 μl Proteinase K solution, and 40 μl Debris Capture Beads were added to the powder. Homogenized samples were lyzed by incubating for 1 h at 55-60° C. with occasional vortexing. A clear supernatant was obtained by centrifugation at maximum RCF for >2 min. To extract the DNA, 200 μl clear supernatant was transferred to a new tube containing 400 μl Binding buffer PN and 20 μl sbeadex beads and allowed to bind for 5-7 min with constant agitation. The beads were spun down and the supernatant removed. The beads were then washed in 320 μl Wash buffer PN1 for 5-7 min while pipetting up and down. The beads were spun down again, the supernatant removed, and washed in 320 μl Wash buffer PN2 with 1-2 μl of RNase. A final wash was done with 320 μl plain Wash buffer PN2. DNA was eluted for 10 min in 55 μl Elution buffer AMP at 55-60° C. with constant agitation.

A first data set of SNPs was created using short reads from all lines of a proprietary pan-genome aligned to the publicly available CS10 reference genome (NCBI GenBank assembly accession: GCA_900626175.2 uploaded on 14 Feb. 2019, submitted by Harvard Department of Organismic and Evolutionary Biology) with minimap2 (version 2.17-r974, options -ax sr and -R to add read-group identifiers, (Li, 2018)). Only unique alignments with a mapping quality of at least 10 were kept. Duplicates were marked with Picard (version 1.140; broadinstitute.github.io/picard/). SNPs were called with freebayes and filtered for a minimal quality of 20 (version v1.3.2-40-gcce27fc, parameters-p 2 --min-coverage 20-g 20000--min-alternate-count 2--min-alternate-fraction 0.2--min-mapping-quality 10 --max-complex-gap-1-b, (Garrison & Marth, 2012)). SNPs were finally filtered for a coverage between 5 and 10,000 within each line and annotated with snpEff (version 4_3t, (Cingolani et al., 2012)).

A second data set of SNPs was created using sequencing data generated by Genotyping by sequencing. Sequence data was processed with Stacks (version 2.5, Catchen et al. (2013)). In brief, reads were processed with process_radtags (options -e apeKl -r -c -q) and aligned to the CS10 reference genome with bowtie2 (version 2.3.5, (Langmead & Salzberg, 2012)). SNPs were then called and retrieved with gstacks and populations (both part of Stacks).

This information was used to create a marker panel through the following process. SNPs from the two data sets were merged to select an initial set of candidate markers 1) with low or moderate effects (always in genes), 2) that are biallelic, 3) that don't occur in regions with high SNP density, 4) that showed variation in the five pivot lines, and 5) that were within regions that could be mapped uniquely to the genome. Within the initial 110,000 candidates, we found about 7,000 rare SNPs (SNPs with a minor allele frequency below 12%). From the initial candidates, the inventors selected about 6,000 that were evenly spaced across the genome. If possible, they selected common GBS-compatible SNPs within a gene of interest. The final set contained about 10% rare SNPs and about 30% GBS compatible SNPs.

The extracted DNA served as a template for the subsequent library preparation for sequencing. The library pools were prepared according to the manufacturer's instructions (AgriSeq™ HTS Library Kit—96 sample procedure from Thermo Fisher Scientific). Targeted sequencing of a custom SNP marker panel based on the Cannabis sativa CS10 reference genome was carried out on the Ion Torrent system by Thermo Fisher Scientific. The library pool was loaded onto Ion 550 chips with Ion Chef and sequenced with Ion GeneStudio S5 Plus according to the manufacturer's instructions (Ion 550™ Kit from Thermo Fisher Scientific).

In a population of 86 individuals of GID:21 001 800, a genome-wide association analysis (GWAS) was performed to detect significant associations between genotypic information derived from targeted resequencing of the custom SNP marker panel described above and the phenotypic variation for the percent total amount (% on dry weight) of varin cannabinoids and amount of total leaf or flower cannabinoids, calculated as ([CBDVA]+[THCVA]+[THCV]+[CBDV]/[CBDVA]+[THCVA]+[THCV]+[CBDV]+[CBCA]+[CBC]+[CBG]+[CBGA]+[CBDA]+[CBD]+[d9-THC]+[d8-THC]+[THCA]+[CBN])*100).

The genotypic matrix was filtered for SNPs having more than 30% missing values within the population and a minor allele frequency lower than 5%. The GWAS was performed using GAPIT version 3 (J. Wang & Zhang, 2021) with five statistical models: General Linear Model (GLM), Mixed Linear Model (MLM), FarmCPU and Blink (model=c(“GLM”, “MLM”, “FarmCPU”, “Blink”). A quantile-quantile plot (QQ plot) was used to evaluate the statistical models. The MLM, which includes population structure and a kinship matrix as covariates thus controlling false positives, performed the best by our evaluation and was used for all further analysis. SNPs surpassing a LOD (−log10(p-value)) value of 3 were considered to have a significant association with trait variation.

The MLM model for log10 percent total leaf varin cannabinoid/total leaf cannabinoid identified a small set of SNPs deviating from the expected p-values on Chromosome NC_044378.1, which is the QTL previously identified by the inventors in UK patent application No. 2102532.5. When evaluating the MLM model for log10 percent total of flower varin cannabinoid/total flower cannabinoid a distinct set of SNPs were identified on Chromosome NC_044373.1 deviating from the expected p-values. Looking only at flower, a QTL that met the specified criteria was not detected corresponding to the QTL on Chromosome NC_044378.1.

The inventors focused on the SNPs from these models showing a strong correlation to the varin trait above the Bonferroni-corrected significance threshold.

On Chromosome NC_044378.1 of CS10 reference genome, the associated SNPs represent a locus of interest between position 66684748 and 70287548, a span of ˜3.6 Mb. Marker common_5002 at position 69028466 on Chromosome NC_044378.1 showed the highest LOD score in the GWAS model evaluated for leaf varin and the QTL, qtIV1, is thus centred around this position (Table 1).

On Chromosome NC_044373.1 of CS10 reference genome the associated SNPs represent a locus of interest between position 5139731 and 47648106, a span of ˜42.5 Mb. The large size of this QTL is most likely is due to linkage drag, however SNPs in this QTL have been still shown to be linked and to demonstrate the ability to distinguish the high-varin trait. GBScompat_common_353 at position 15729253 on Chromosome NC_044373.1 showed the highest LOD score in the GWAS models evaluated for flower varin and the QTL, qtIV2, is centred around this position (Table 2).

Follow up experiments using a F2 population GID: 21 002 073 derived from a cross between siblings that were the progeny of GID: 20 000 110 0000 and 20 000 434 0000 were used to demonstrate that the high varin trait could be introduced into a plant population of other genetic backgrounds and followed. The segregation pattern of the high varin trait in this population followed the pattern for a monogenic trait segregating in a 1:2:1 ratio. A GWAS assay on this population was carried out as described above using leaf tissue for the cannabinoid assay. Here the inventors identified two additional associated SNPs at positions within qtIV1 with LOD scores above the Bonferroni-corrected significance threshold that are predictive of the high varin trait, designated as rare_214* and common_1780* (Table 1). This independently verifies qtIV1 as well as demonstrating that this QTL is independent of tissue type.

These genomic regions represent two QTLs with the highest likelihood of containing the genetic element responsible for the high-varin trait, designated as qtIV1 on Chromosome NC_044373.1 and qtIV2 on Chromosome NC_044378.1. The strong correlation between cannabinoid in leaf and flower shown in FIG. 2 supports our conclusion that qtIV1 and qtIV2 are responsible for the high varin trait independent of tissue type.

The SNPs identified for the high-varin trait are predictive. Within the population, 20 001 800 0000, for each region of interest, every potential allele state for every targeted SNP was determined and assigned as homozygous for allele 1, homozygous for allele 2, or heterozygous. For each allele state the average leaf or flower percent total varin was determined ([CBDVA]+[THCVA]+[THCV]+[CBDV]/[CBDVA]+[THCVA]+[THCV]+[CBDV]+[CBCA]+[CBC]+[CBG]+[CBGA]+[CBDA]+[CBD]+[d9-THC]+[d8-THC]+[THCA]+[CBN])*100) from the plants in the population that contained each allele state. Therefore, plants that contain the allele state, which is predictive for high percent total varin trait, have higher varin content, and each predictive allele state can be associated with a higher varin content.

Tables 1 and 2 below include the allele positions one could use to identify the presence of one of the high-varin QTLs and thus determine a high-varin trait, either by marker resequencing as is described herein, or by PCR methods known in the art. In Table 2, four additional SNPs showing LOD scores under 3 were included to demonstrate linkage decay away from qtIV2. As linkage decays the SNPs cannot predict the high-varin trait.

TABLE 1
SNPs associated with the high-varin trait on chromosome NC_044373.1,
defining qtIV1. The presence of the high-varin trait is predicted by
the occurrence of identified alleles as homozygous for allele 1 or
homozygous for allele 2. Asterisks (*) next to allele 1 or allele 2
indicate that this allele determines the presence of the high-varin
trait when in a homozygous state. The positions of the SNPs are
provided with reference to the CS10 reference genome as described
herein. “Homo_1” denotes the homozygous allele 1 percentage (%)
varin, “Homo_2” denotes the homozygous allele 2 percentage (%)
varin and “Hetero” denotes the heterozygous percentage (%)
varin from CA.

| | Posi- | | Allele | Allele | | | | Context sequence |
|---|---|---|---|---|---|---|---|---|
| SNP | tion | LOD | 1 | 2 | Homo_1 | Homo_2 * | Hetero | (cs10 reference genome) |
| GBScompat_ | 15729253 | 5.84 | A* | T | 0.17181096 | NA | 0.05891922 | GACACCCCTGATCATATCTTTCATT |
| common_ | | | | | | | | CACCCTATTTTTTAGTTGAAACAAC |
| 353 | | | | | | | | ATAAATTTTAAATGTGGTGTGTTCC |
| | | | | | | | | CAAGCAGTAAATGTCTAAAATTACA |
| | | | | | | | | GGGAAACCCAAAACCAATTCTAGAC |
| | | | | | | | | GTAAATCTGGTGTTTTGGGTTGAAG |
| | | | | | | | | TACTCAACTTTCGAAAGGGTCGACT |
| | | | | | | | | CTGATTGAGTTGACTGGATACACAG |
| | | | | | | | | C[T/A]GCAGCGATTCTCTCCCTGA |
| | | | | | | | | GGTTAA |
| | | | | | | | | ATTTCTTCGCAATCAGCGGTCGCAC |
| | | | | | | | | ATCATCAACTCCAACTAAGCTTTCC |
| | | | | | | | | AAAAATGGAAGCAATGAAGGCAACT |
| | | | | | | | | CTTTATCCAAAGGATCATCGAACCA |
| | | | | | | | | GCTTTCAATTGGTACCCCATTGTCC |
| | | | | | | | | ACTTGAAATCCAAATGCCTGAAAGA |
| | | | | | | | | GACATAGTAATCAAATTTTCTCCCA |
| | | | | | | | | A |
| | | | | | | | | (SEQ ID NO: 1) |
| common_ | 13591184 | 5.35 | G | A* | 0.05794687 | NA | 0.16953522 | AAGATTCTGAATGTACAATGAGAGA |
| 1811 | | | | | | | | TTAAAATCTTGAGATCATCTTCGCC |
| | | | | | | | | TCCACCAGTCTGGAATTTTTAGCCT |
| | | | | | | | | TTGGTTTCTATGTACAGCAAGTTTA |
| | | | | | | | | ACCCCAGGAAGCTTATGTTTCACCT |
| | | | | | | | | GTCTCCATAACATATCAACTGCATC |
| | | | | | | | | TTCCTTTTTCGGTGGGTACTGGAAT |
| | | | | | | | | TCAAAATGATGCGAAATGTTCTTAA |
| | | | | | | | | G[T/C]TGTCTCCACATTTCAACCC |
| | | | | | | | | ATTTTT |
| | | | | | | | | CCTTGGGAAATTCACGGAGCTTAGT |
| | | | | | | | | AACCATGTAACCAGGTTCCAGAGCC |
| | | | | | | | | TCTTTAAATGAGAAGAATAATGAGA |
| | | | | | | | | ACTGGCTGTAGTCAATTTCATCCTC |
| | | | | | | | | GAATGGGAGCTCAATTTGATCACTC |
| | | | | | | | | ACTATGACAGGAATACAATGACTCA |
| | | | | | | | | CAATAGCATCAAATAAACGACATGA |
| | | | | | | | | T |
| | | | | | | | | (SEQ ID NO: 2) |
| common_ | 39975423 | 4.18 | G* | A | 0.17537548 | 0.0555699 | 0.11038804 | AACATGCTAATTCTGATTTAAAGCT |
| 2008 | | | | | | | | GCATATTTGGTTTAGAGATATATTA |
| | | | | | | | | CCTGGGCAAGTTTCAGATTTGTACA |
| | | | | | | | | TTTAAAGCAAATCTGGTGGATAATG |
| | | | | | | | | TAAAAGGAAATTTGAAATACATTGT |
| | | | | | | | | AACTCCAGTCTGTGAAACTCACATT |
| | | | | | | | | TTACATTGACATGTAATGCTCTTTG |
| | | | | | | | | TTTTCATACTTGAAGGAACAAGAAG |
| | | | | | | | | G[T/C]GCGACTTTGCTTGTATCTG |
| | | | | | | | | CAAAAT |
| | | | | | | | | TTGATGTAGTTTCTGTCCGTAATAT |
| | | | | | | | | CTACCTTCAGTTTGAAGAGGTATGT |
| | | | | | | | | TTTTCTTCTTTTGTGAAGCATGACT |
| | | | | | | | | TTCAAAAATTTCAAGTTTAAATGTG |
| | | | | | | | | TGTGGTTGTTCCCTCTACATGGTTC |
| | | | | | | | | TCTGTTTGATGTACCATTACTTAAT |
| | | | | | | | | CTGGCCTGCTCTATTGAGTCATATT |
| | | | | | | | | T |
| | | | | | | | | (SEQ ID NO: 3) |
| GBScompat_ | 32238016 | 4.15 | A* | C | 0.17716198 | 0.05936794 | 0.1109979 | TTCGGAGTTGGACCAAGATGAATGT |
| common_ | | | | | | | | AGTGTTTATTTGCAGCAGAGTAGCC |
| 374 | | | | | | | | CTCCTATTAACTCAATCACTGGCTT |
| | | | | | | | | TTCTGGTATGGTTTTTCTATCACTA |
| | | | | | | | | GGAATCATGCTTGACCCAATTTATG |
| | | | | | | | | GGCAGACAAGTTATATTTATCTTGT |
| | | | | | | | | CTAACATAAAATATTCTAACTGGCT |
| | | | | | | | | CAGTTTCACTCGGAGCAATTACATC |
| | | | | | | | | T[T/G]CTGCCGTAGATAATGGGAG |
| | | | | | | | | TACTAT |
| | | | | | | | | AGCTGCTCAGAGTGCAACACAAAAT |
| | | | | | | | | CCATCCCTGGAAGCTGCATTTCATC |
| | | | | | | | | ACGGGATATCTTCTAGTGTTCCTAA |
| | | | | | | | | CAGCTTATCCTCTCTAGTTAGAATT |
| | | | | | | | | GAATCTCTAGGCAATCATGCTGGCC |
| | | | | | | | | TTTCAGAATCCAATCATTCATCGGG |
| | | | | | | | | GCCACTAAAGTTTGACATCCATGGA |
| | | | | | | | | A |
| | | | | | | | | (SEQ ID NO: 4) |
| common_ | 13750613 | 4.12 | A* | T | NA | 0.05732232 | 0.1705918 | CTGACCTTTTCTTTTTTCCTTCTTT |
| 1813 | | | | | | | | CCGGTGTATGAAGGTGGAAACCATT |
| | | | | | | | | ATCAAGAAAATGGAAGTTACTGGAG |
| | | | | | | | | GCTAAAACCATCTTGGTTTTATGGC |
| | | | | | | | | ACTGATCGGTATGTTTTTCTTTTTC |
| | | | | | | | | TAAGACTCTTAATCTCCATCAATAC |
| | | | | | | | | TGATGAATTTATACAGCTTTTATTT |
| | | | | | | | | ATTTATTATGCTACTGTGTTTGTTT |
| | | | | | | | | T[T/A]ATAGGACTTTGGCAAATGT |
| | | | | | | | | GTGGAG |
| | | | | | | | | GGCGAGACGCTACTGTTATCTTACA |
| | | | | | | | | AATTAGTTTAAATAAAAGAACAAGA |
| | | | | | | | | ATCAGTTTTTCTAAGAGCAGAACCT |
| | | | | | | | | CTGGTATGGAATTTGAATCAGAGCG |
| | | | | | | | | GAGGAAACACAAGAAATCGTTGGCA |
| | | | | | | | | TTTACCACTGAAGTTATTCGTTCTG |
| | | | | | | | | CAACATTTTTTATGGCTTGGTCTTG |
| | | | | | | | | T |
| | | | | | | | | (SEQ ID NO: 5) |
| common_1939 | 32285533 | 4.11 | G | A* | 0.05936794 | 0.17501793 | 0.10918487 | TTCAGATTAAAGCCGGTGCCAAGCT |
| | | | | | | | | TTTTCTTTACGACTTTGATGTGAAG |
| | | | | | | | | CTTCTTTATGGTGTCTACGAGGCCA |
| | | | | | | | | CTTCAGTTGGTGCTCTCAACTTGGA |
| | | | | | | | | ACCCACTGCCTTTCATGGAAAATTC |
| | | | | | | | | CCTGCCCAGGTACCTCCCCTCTTCT |
| | | | | | | | | TCGTCTTCTTCTTTAAGGGTCGGTT |
| | | | | | | | | TTGTTTTGATTCCTTGTTCGTTTGT |
| | | | | | | | | T[T/C]AGGTCAAGTTCAAGATTTT |
| | | | | | | | | CAAGGA |
| | | | | | | | | ATGTTTACCTCTTCCCGAGAGGGTT |
| | | | | | | | | TTCAAAGCTGCAATTATTGACAATT |
| | | | | | | | | ACCAGGGTTCAAGGTTTAAACAACT |
| | | | | | | | | ACTTAGTAGTGCACAGGTAAAGCTA |
| | | | | | | | | CTACTTGATTTGTAATCTCGTTTAT |
| | | | | | | | | CATTATTATTAATTGAGTTTATTTC |
| | | | | | | | | TTTCTCAATTCAATTCAATGAAGGT |
| | | | | | | | | G |
| | | | | | | | | (SEQ ID NO: 6) |
| common_ | 39909542 | 4.11 | G | A* | 0.05936794 | 0.17501793 | 0.11145163 | GCCATTAAGCCCAATGCCTCCATGG |
| 2007 | | | | | | | | ACTGCCATTTTCCGAGCACCAATGC |
| | | | | | | | | AGCATGCTGCATGAGCCCCACGGTG |
| | | | | | | | | GGGCGGGACAATGGATCCCACATCA |
| | | | | | | | | AGCAGCTTCCATGACAATGTTATAC |
| | | | | | | | | CGAGAGTCTCGTGGCATGATAATTG |
| | | | | | | | | TCCAATCCATGTGTCATTCATACGG |
| | | | | | | | | TTCCCATGATCATCAATTCCTCCAA |
| | | | | | | | | A[A/G]ACAACTAGAAGTTCACCAA |
| | | | | | | | | TT |
| | | | | | | | | ACAACACATGAATGTCCAAATCTCC |
| | | | | | | | | CACTTGGAATGCCTGAATTAAGCTT |
| | | | | | | | | CTGCCACTTCAACTTTCTTTGACAA |
| | | | | | | | | TCATTACCAATATATGCCACCCATG |
| | | | | | | | | TGTCATCAAGATGGCGTCCTAAACA |
| | | | | | | | | GTAAAAGAGAAAAGAGAAACAAAAT |
| | | | | | | | | CAATGCATAACAAAAAGAAAATTAA |
| | | | | | | | | GAGCA |
| | | | | | | | | (SEQ ID NO: 7) |
| common_ | 47648106 | 4.09 | G* | A | NA | 0.05825239 | 0.17519094 | TCTTCAATCTTCAATCATAGTAGAG |
| 2060 | | | | | | | | AATAATAGATACAACATAAATATTA |
| | | | | | | | | TGGCTTGTTTTGTGTTTGGTGTATG |
| | | | | | | | | ATGATAATAATGATGTTTGACGATT |
| | | | | | | | | AATAAAACACAAGCCTAAAATGGAG |
| | | | | | | | | TTGAGAGAGTGATCATGATGGAAAT |
| | | | | | | | | AATATTAATCAATCAAACATCTCTG |
| | | | | | | | | TCACGTTTGCACTCAGGTGACATGT |
| | | | | | | | | T[A/G]GGGTATCTGACACGATCAG |
| | | | | | | | | TGCAAT |
| | | | | | | | | AGTTGTAAATGGTGTACTTTTGTCT |
| | | | | | | | | GACCCACCTGAGATACCTCCACTGG |
| | | | | | | | | CTCTGGTCAAGGTCCTGGAACTCCT |
| | | | | | | | | TACCATCCCACCACCTCTTTCCTTG |
| | | | | | | | | GGTGGCACAGTACTTGGCTTGAACC |
| | | | | | | | | GATGACTCGCACCCATCGATGTGGA |
| | | | | | | | | ACCCTCTGTAGGCCGCTATGAATGG |
| | | | | | | | | G |
| | | | | | | | | (SEQ ID NO: 8) |
| common_ | 5139731 | 3.97 | G* | A | NA | 0.06063646 | 0.16643811 | TATTTTGATTTGGGAATTTTTGATT |
| 1758 | | | | | | | | ATTTGTTCTGACAAATTTGAAATCT |
| | | | | | | | | TCGTTATGGAGCAGGAATCAAGCAA |
| | | | | | | | | AGTGTTAAGCATGTCTAGAGTTCGC |
| | | | | | | | | TGCATTCTCCGTGGTTTGGATGTGA |
| | | | | | | | | AAACTCTTGTCTTTCTCTTTGCCCT |
| | | | | | | | | TATCCCAACTTGCATCTTTTTCATC |
| | | | | | | | | TATGTTCACGGACAGAAGATCTCAT |
| | | | | | | | | A[C/T]TTCTTGCGGCCACTGTGGG |
| | | | | | | | | AATCAC |
| | | | | | | | | CACCTAAACCTTTTCATGATATGCC |
| | | | | | | | | GCACTATTATCATGAGAATGTGTCA |
| | | | | | | | | ATGGAACATCTTTGTAAACTTCATG |
| | | | | | | | | GTTGGGGAGTGAGGGAGTATCCTAG |
| | | | | | | | | GCGTGTTTATGATGCTGTGTTGTTT |
| | | | | | | | | AGTAATGAGCTAGACATCTTGACCA |
| | | | | | | | | TTCGCTGGAAAGAGTTGTATCCCTA |
| | | | | | | | | C |
| | | | | | | | | (SEQ ID NO: 9) |
| common_ | 32332871 | 3.91 | G | A* | 0.0574302 | NA | 0.17519094 | ACTTCTAAAAATGGCGGCATCTTCA |
| 1940 | | | | | | | | AGACTAGTGCTGCATCTACATGCCA |
| | | | | | | | | CAACCACCGCAGCTATAGTGGTGCC |
| | | | | | | | | TACACCCAAGTACAACCTTAGATTA |
| | | | | | | | | TCCACCGCCACAGCTGCTAATCGCC |
| | | | | | | | | GCTTTCGAAAACCCATATTCAAATG |
| | | | | | | | | TAAGGCTACCTCTAACACTACTCCT |
| | | | | | | | | ACTTCTACTCCTGTTTTCCAAGGAA |
| | | | | | | | | T[C/T]TACGGTCCTTGGTCCGTCG |
| | | | | | | | | ATTCCA |
| | | | | | | | | CCGACGTTAGAGAGGTCATATCCTA |
| | | | | | | | | CCGTTCTGGGCTGGTCACAGCTGCA |
| | | | | | | | | GCCTCTTTTGTTGGGGCAGCCTCCA |
| | | | | | | | | CAGCTTTCTTGCCTGAAGAAAATCA |
| | | | | | | | | GGTCGGGGAATTCATACACCACAAT |
| | | | | | | | | CTTGACCTGTTTTACATTGTGGGTG |
| | | | | | | | | GTGCTGGACTTGGGTTGTCTTTGGC |
| | | | | | | | | T |
| | | | | | | | | (SEQ ID NO: 10) |
| common_ | 6632869 | 3.80 | A* | C | 0.16768549 | NA | 0.05755709 | TCATGGTTGTGTGATTAAATTTTAA |
| 1777 | | | | | | | | TAATTAAATAAATACTATATTTGAT |
| | | | | | | | | GTGATTACTAAATTGGATCAACATA |
| | | | | | | | | TCACCTACATATAGTTTGTATGTTT |
| | | | | | | | | AAAAAATTAATACTAGAGAAATTAG |
| | | | | | | | | ATAGGAGAGATATAATTTTAATGTA |
| | | | | | | | | AATGTGTACCTGATAGCTTCCAATA |
| | | | | | | | | ACATGGATGACGACAAACATATTAG |
| | | | | | | | | C[G/T]GAAGCAATAAGCCAAGCAG |
| | | | | | | | | GCTTTT |
| | | | | | | | | CAAGGGTGATGAGAATATTGTCATC |
| | | | | | | | | TACCGAGTTACCAAAAACATAATAA |
| | | | | | | | | CCTATCAAAGCAACTGGAAAATAAC |
| | | | | | | | | AAAGAGCTACTACTATGTAAGCCAC |
| | | | | | | | | AACTACTCCTCTCCACATTGGTTTC |
| | | | | | | | | TTAGATGGTTTTTCTGGTGTGGATG |
| | | | | | | | | GGATTGTGGCTTGAATCTCAAGCAC |
| | | | | | | | | C |
| | | | | | | | | (SEQ ID NO: 11) |
| GBScompat_ | 8039846 | 3.75 | A* | T | 0.16860398 | 0.05591285 | 0.11334089 | CTCAAAACTCCACTTTCTGCTGCTC |
| common_ | | | | | | | | GACATGTTATCATTGAAACCCACTA |
| 346 | | | | | | | | CTACACCTCTCAACAACAACCCCAA |
| | | | | | | | | CATCTCCGGCGACCGGAGTTTCAGC |
| | | | | | | | | TCGATCTCCACCGCCACCGCCGGGA |
| | | | | | | | | AAGATGGAGACCTTAGAAGAAAGAC |
| | | | | | | | | CCGCGTGGCGGTTTCTGGGTCGAAA |
| | | | | | | | | CTCAGGCGACGTGGGTCGGTTCGGG |
| | | | | | | | | C[A/T]GCGATCAGTAGTGGGGACA |
| | | | | | | | | ACAAAA |
| | | | | | | | | CAGAGACTGTGAGTAGTAATAGCTC |
| | | | | | | | | TGTTCCGGCTCATCAGAGTGAAGAT |
| | | | | | | | | AATTCCAACGGTTCTCTGAAGAAGA |
| | | | | | | | | AGAAGCCATCTAAAGGAATTGAAGT |
| | | | | | | | | TAGAGCAGTGATGACTATCAGGAAG |
| | | | | | | | | AAGATGAAGGAGAAG CTCGCTGAA |
| | | | | | | | | AAAATGGAGGATCAATGGGAGTTTT |
| | | | | | | | | TC |
| | | | | | | | | (SEQ ID NO: 12) |
| common_ | 3375992 | 3.70 | G | A* | NA | 0.15998989 | 0.06278542 | CACAATATTACAACATGTACAGTTT |
| 1735 | | | | | | | | GTGCTATAAGTTTCTATCTTTTTTC |
| | | | | | | | | TTCTTCTTCTTTTTCCTTTATTTTT |
| | | | | | | | | AGGCCAAAACTAAACATGGTAGATC |
| | | | | | | | | ATCCCCACCTCGAGAGTGGAAGCTC |
| | | | | | | | | GGGGCACGTTTAGATCATAAGTGGC |
| | | | | | | | | TCCTCTGCTGTTTCTTCTCAAGAAC |
| | | | | | | | | TCGTATCCATAATCGATTGCAATCC |
| | | | | | | | | T[C/T]TTCGTTATACCTGATCCGG |
| | | | | | | | | GATTTG |
| | | | | | | | | CTCTCATGTAAGTGTTCCCTAATAT |
| | | | | | | | | GTAAGCTATTCCGGTTTCCTTAGCT |
| | | | | | | | | TCCATCAACTCTGCGAGTTCTGCCC |
| | | | | | | | | TCATACTCGTGTCAATCTTGGGACT |
| | | | | | | | | CTCCGGCAAAACAAACCTCACCTTC |
| | | | | | | | | TTTTTTCTTGGAATAGCTGGTGGT |
| | | | | | | | | GATGGTGATGTTATCTCTCTATGAT |
| | | | | | | | | TT |
| | | | | | | | | (SEQ ID NO: 13) |
| common_ | 4235438 | 3.51 | G | A* | 0.0591001 | NA | 0.16363803 | GAAAATATGTGGTTTTTTTGTGTAT |
| 1746 | | | | | | | | AACTTTGTTTAGTATCTCCAAGATC |
| | | | | | | | | CTATGTTGGTCTTTAACAAAGAAAC |
| | | | | | | | | ATATAGTAACTCATTCCAAGTATCT |
| | | | | | | | | GGAACACCAGGCAAACACCAATGGC |
| | | | | | | | | TGCAATCTTGAGAATGTACAGCAGC |
| | | | | | | | | AATTTGCTCCTCTACTGATTTATAT |
| | | | | | | | | TCCATTCGATAAATCGAAGGGTGAC |
| | | | | | | | | C[A/G]TCTTTTCTGTAATCTGTAA |
| | | | | | | | | GCCTGC |
| | | | | | | | | TTATGTTTAGATATGTCACTGGAGT |
| | | | | | | | | TTTCATATCTTGAATCACATACTCT |
| | | | | | | | | AAAGCCCTCATCTTTTTATTGTACT |
| | | | | | | | | TAGCTAAATAAGTGTTGTTGAAAAT |
| | | | | | | | | CGGCTCGGTTTCTTTGTGGCATTGT |
| | | | | | | | | CCTCCTGAGTTCCATGGCCCTCCTC |
| | | | | | | | | TGCCAACAAGATTAAAGTTCAACAT |
| | | | | | | | | T |
| | | | | | | | | (SEQ ID NO: 14) |
| common_ | 32711414 | 3.30 | G | A* | 0.05626831 | 0.17537548 | 0.11076794 | AATTCTGATAATTACTTAGCACATA |
| 1945 | | | | | | | | GAGAATAATAAGAATTGCCAGAAAT |
| | | | | | | | | GTTGCCCATGTTTGATCCAAACGAC |
| | | | | | | | | AATGAAGCTGGTATGAAGCTTTTGG |
| | | | | | | | | AGGACCTAACCACAAATGCACACCA |
| | | | | | | | | TTTTCAACAACAGGCACTGAAGGAG |
| | | | | | | | | ATACTATCAAACAATGCTGCCACTG |
| | | | | | | | | AATATCTAAGCAGCTTTCTCAATGG |
| | | | | | | | | T[C/T]ACTCTGATATGAAGCTTTT |
| | | | | | | | | CAAGGA |
| | | | | | | | | AAGAGTTCCCATTGTGAAGTATGAA |
| | | | | | | | | GATATCAAGCCTTTTATCAACCGAA |
| | | | | | | | | TTGCCAATGGGGAATCCTCCAACAT |
| | | | | | | | | CATTTCAGCTCAACCAATAACAGAG |
| | | | | | | | | CTTCTTACGAGGTATAACATCACAT |
| | | | | | | | | ATATATATATATATATATATATGA |
| | | | | | | | | TATATGACATGACATGTTATGACAC |
| | | | | | | | | AT |
| | | | | | | | | (SEQ ID NO: 15) |
| common_ | 39334764 | 3.18 | G* | A | 0.17384844 | NA | 0.06051052 | TAGATATACTTGAATAATATACCGT |
| 2000 | | | | | | | | GCTCCATGTATAGCTAGCTCTTTCA |
| | | | | | | | | TTCTGGCTATAAACTAATTGAGGGG |
| | | | | | | | | ATAATACATATATTATATATACATA |
| | | | | | | | | TTTTGTATACTTATCTTCTTTCATT |
| | | | | | | | | GTTAACGAAAATGAGGATTAGGGAT |
| | | | | | | | | GTGTTATTGGGTGCATTGGTGAGCT |
| | | | | | | | | ATCTGATCATACAAAACGTTTGTGT |
| | | | | | | | | A[G/A]CAAATGCATCGAGGGTTTT |
| | | | | | | | | ACAAAA |
| | | | | | | | | GTCACAGTTTTTTGCCAACTTTTTG |
| | | | | | | | | CAATCTAATAATAATGGGATTAGTA |
| | | | | | | | | ATAATGGGACCAAATGGGCAGTTCT |
| | | | | | | | | TGTTGCTGGCTCCAATGGCTGGGGT |
| | | | | | | | | AACTACAGGCATCAGGTATTTAATG |
| | | | | | | | | GGACACCATAAACACGGGAGGGAGT |
| | | | | | | | | ATATATAAATATACATATATATATA |
| | | | | | | | | T |
| | | | | | | | | (SEQ ID NO: 16) |
| common_ | 38186703 | 3.05 | A* | C | 0.17305256 | NA | 0.06160463 | TATTTCACAACAAAAAGACACGAGA |
| 1987 | | | | | | | | AAAATTGTCTAATCAAATCAAATTC |
| | | | | | | | | CAGTATCATGCTAGTATTAAGTGAA |
| | | | | | | | | TCAAAAAATCAAGAGGCATATAATA |
| | | | | | | | | TATAATAGAGAACTAAGAGAAGCAC |
| | | | | | | | | AATAAAAGAAATATCAGCACAATGA |
| | | | | | | | | TAGTTAGAGTAGCTAAATTTGAGAA |
| | | | | | | | | CATATCAGATGTCAATGAAAATAGG |
| | | | | | | | | A[A/C]ATTGGCGCACCTTCTCCAC |
| | | | | | | | | ATAGCT |
| | | | | | | | | GTTCAAATCGAGCAACTATGTGTTT |
| | | | | | | | | TCCATACGTATATTTCCTCAGAGCA |
| | | | | | | | | GCAAGATGGGCTCTTGTGCGATTCA |
| | | | | | | | | GCAATATTGCTCGCTGTCTGTCATT |
| | | | | | | | | GCTTTTCTCAAACATCTTTTGCACC |
| | | | | | | | | ACATAATTAGCAAATTGGTCCTTCA |
| | | | | | | | | TCATTGTCTAGATATAGGAAGCAAA |
| | | | | | | | | C |
| | | | | | | | | (SEQ ID NO: 17) |
| rare_ | 29989266 | 6.22 | A* | C | 43.80 | 29.50 | NA | AAAGGCAAGGATTGAAATGAGCAGA |
| 214* | | | | | | | | ACAGGACCAGTGAACAACAAGAAGA |
| | | | | | | | | AGCTTCCTGCAAATTATAGCACCAA |
| | | | | | | | | GTATAAAGAACGCAAAAGTTCCATC |
| | | | | | | | | AAATAAAAAAAAATAGTGTGAAAAG |
| | | | | | | | | GGACAGAAAATTTGGAAATCAAACC |
| | | | | | | | | TGTAAACCCAAAAACAGTTCCACCA |
| | | | | | | | | GTTGCATTGAACAATACAGTGATAG |
| | | | | | | | | T[G/T]TGAGTTGAAAACTTTCCTG |
| | | | | | | | | GCAACA |
| | | | | | | | | AAAATGCAATACCAACCCATAAAAT |
| | | | | | | | | AAAAACGACCCACATTAGAACTTTG |
| | | | | | | | | AGTATGAATTTGGCCAAAGAGGTAA |
| | | | | | | | | AAAGAGATGTTTTCCTCTTGACATA |
| | | | | | | | | ACTAGGCTCCACAGTCGGAGGCAAC |
| | | | | | | | | AGAAGAGGCTTCTCAACAGAGTTTC |
| | | | | | | | | CGTCCATGGCGAAAACCTTACACAA |
| | | | | | | | | T |
| | | | | | | | | (SEQ ID NO: 18) |
| common_ | 6838024 | 5.91 | A | G* | 5.29 | 38.93 | 52.41 | CCATCCGACACTCCTCAACGTTCTG |
| 1780* | | | | | | | | AGGAATGGTTTGCCCTTCGTAAGGA |
| | | | | | | | | CAAGCTAACCACAAGCACTTTCAGC |
| | | | | | | | | ACTGCATTGGGTTTTTGGAAAGGAC |
| | | | | | | | | AGCGTCGAATGGAGCTTTGGCGCGA |
| | | | | | | | | GAAGGTGTTTGCATCAGAGGTCAAA |
| | | | | | | | | ATCATACAAGGTGCACAAAGATTTG |
| | | | | | | | | CTATGGATTGGGGTGTTCTCAATGA |
| | | | | | | | | A[G/A]CAGAAGCTATAGAAAGGTA |
| | | | | | | | | CAAAAG |
| | | | | | | | | CATTACAGGCCGGGAAGTTGATTCG |
| | | | | | | | | CTAGGATTTGCTGTTCATGCTGAGG |
| | | | | | | | | AGCGATACAATTGGGTTGGCGCCTC |
| | | | | | | | | TCCTGATGGTGTTATTGGATGCTTC |
| | | | | | | | | CCGGAGGGTGGAATTCTGGAAGTGA |
| | | | | | | | | AGTGTCCTTATAACAAGGGTAAGCC |
| | | | | | | | | TGAGTTGGGATTGCCTTGGTCTAAA |
| | | | | | | | | A |
| | | | | | | | | (SEQ ID NO: 19) |

TABLE 2
SNPs associated with the high-varin trait on chromosome NC_044378.1, defining
qtIV2. The presence of the high-varin trait is predicted by the occurrence of identified alleles
as homozygous for allele 1 or homozygous for allele 2. Asterisks (*) next to allele 1 or allele
2 indicate that this allele determines the presence of the high-varin trait when in a
homozygous state. The absence of an asterisk indicates these SNPs are not able to predict
the high-varin trait. The positions of the SNPs are provided with reference to the CS10
reference genome as described herein. “Homo_1” denotes the homozygous allele 1
percentage (%) varin, “Homo_2” denotes the homozygous allele 2 percentage (%) varin
and “Hetero” denotes the heterozygous percentage (%) varin from CA.

| | Posi- | | Allele | Allele | Homo_ | Homo_ | | |
|---|---|---|---|---|---|---|---|---|
| SNP | tion | LOD | 1 | 2 | 1 | 2 * | Hetero | Context sequence (cs10 reference genome) |
| common_ | 69028466 | 5 | A | G* | NA | 0.083 | 0.19 | GTCAATATTTTTAATTTTTATGTATACATAATATTAGATTTAG |
| 5002 | | | | | | | | TATGCAAATTCATGTAAGTTATTATTAATTTAGACAATTATAT |
| | | | | | | | | ATATTTATATATATATATATATGATATGTTCTTACACTAATTG |
| | | | | | | | | AAGAGCCATGGTGGGATCTTGGCGTGAGTAGTGATTGTTA |
| | | | | | | | | TTTGGTTGTAAAGCATTGACTTGGAAATAATT[T/C]CCTGGT |
| | | | | | | | | TGCTGGTGTACCACAACTGATGCTGCACCCTCATCATCATT |
| | | | | | | | | ATTATTATTATTATTATTATTATGATCATTGTTATTATTATGA |
| | | | | | | | | TTTTGATGAATTAGTTCATGATGATGACCATAGCTGCTTGT |
| | | | | | | | | ACCAACATTCATGTTCATATTTATGTTCATGTTCATATTCAT |
| | | | | | | | | ACTCATGCTTTGGTTCCTCTCATTCTCA |
| | | | | | | | | (SEQ ID NO: 20) |
| | | | | | | | | GCTTAAAAATCTATATATTGAAAAAAAAAAAAACATGAATTA |
| pooled | 68813383 | 4 | G | A* | NA | 0.08 | 0.19 | AAATTTATAATTACAGGGATGTAACATTTTCTTATTGATAAA |
| Seq_7 | | | | | | | | TCCCAAAGTTTCAATTTTTTTTTTTAATTTTAAATTTTCTCAT |
| | | | | | | | | TCTAATAATAAAAAATAAAAAATAAAAACTTTTAATTATGCTT |
| | | | | | | | | CCAAGAAACCAGTCATTCTAAGAATAGAATT[C/T]CTAACAT |
| | | | | | | | | GACTCAAATCTCTCCCCTTAAGAGTTCTTCCATTGCCCGAA |
| | | | | | | | | CAAACCGAAAAAGCCATCTTTCCACCGGTTAACCGCCTCC |
| | | | | | | | | TGACGATGACATGAGGCGGTACCAGTTCTTCATTTTCATCA |
| | | | | | | | | TAATCATAATCATAATCATAATTATCATCATCGAAATTATTC |
| | | | | | | | | CAAATTTCGTGATTTAAATGATCATTAATA |
| | | | | | | | | (SEQ ID NO: 21) |
| common_ | 68477632 | 4 | G | A* | NA | 0.104 | 0.173 | TTGATACAGAAGTTTGTCAAAGTAGTTTACATACATACATAC |
| 4995 | | | | | | | | ATACATTGATAAAGGAAAAGCTATAGTAGTAGAGCCATGAA |
| | | | | | | | | AACTGGTAGTAACACTGGGGATTTACCGGGTTATATCAGG |
| | | | | | | | | CGTTTCAAACCCGTGATAGAAGCATTCGGTTTTTCCTCGAA |
| | | | | | | | | AAGTTCAAGTTCAAGTCATCCTGCTTAAATCTACTCT[C/T]G |
| | | | | | | | | TTCCTCCTCCCCATTGTCGTTCTTTCCTCGTTTTGAATTCTA |
| | | | | | | | | CGTCGATAAGGAAGTAACATTTGTCATTCTCTCTAATGTAC |
| | | | | | | | | TTGATCTGTCCATTCATTCTACTGAGAAGTTTTCGAGAAAG |
| | | | | | | | | GTTTAACCCGAGCCCTTCTTGTGAAGTCCAGTGCTTTCCAC |
| | | | | | | | | TCTCAACCATGTCTTGGATAAGAGCATTAGGAATA |
| | | | | | | | | (SEQ ID NO: 22) |
| common_ | 66684748 | 2 | A | G* | NA | 0.112 | 0.252 | ATAGAAGCATTACTTCTTGCTTCTGAGTCGAGAATCGAAAAG |
| 4973 | | | | | | | | TCCAGCAAAGAAATTGATCTCAGTGCCAACTTGGTCACCAAT |
| | | | | | | | | GATCTGGACTCCACGGCTGAAGCTAATCTTGCATTTAGAAG |
| | | | | | | | | ATTCAAGCAATTTGGTCGTGGCAATTCTCAACTCAGCAATGC |
| | | | | | | | | TTCTGCTCAATTTAACAGGATTCCTAATCCTAAT[A/G]TCAGG |
| | | | | | | | | CTCAACAATTTTTCTCCCAATCCCAATCAGAGTAGCATGAGC |
| | | | | | | | | AGAGGTAATTTCAATTTTAATCCTCTCAAAAATAACAGGTTTG |
| | | | | | | | | GATTTACCTTTCCTAACCGGCCTCAGTGCCAACTCTGCTTGC |
| | | | | | | | | GATTTGGTCATGTTGTGCAAGATTGTCCCTTTCGTTTTGACA |
| | | | | | | | | AATCTTTCTCAGGACCACCTTTAGCTA |
| | | | | | | | | (SEQ ID NO: 23) |
| common_ | 67434963 | 2 | A | G | 0.121 | 0.161 | NA | TTTTTGTCGTTGAAAATTCTCCCTGAACTATAATTAAGTGATT |
| 4981 | | | | | | | | AGCATTGCATTAGGCATCAGGAAGGTGATCATCTCTCTCCG |
| | | | | | | | | TACCTTTTATTTCTCTATTATGAGGGTCTTACTAGTGCCCTTA |
| | | | | | | | | AGATTCATGAAAGGCTTGGTACGTAGTCTCATGGGTATCTTT |
| | | | | | | | | GTTGCTCGCACTGCCTCTGCCATTTCTCACTT[A/G]CTTTTTG |
| | | | | | | | | ATGATGACAATCTCCTATTCACCACTGCTACCCATACTTCTTT |
| | | | | | | | | CAATGCTTTGGAGAATGCCCTTATTCTTTATAACCTAGCCTC |
| | | | | | | | | TGGTCAAAAGGTTTATTATGGGAAGTCTTCCATTTTGTTCTC |
| | | | | | | | | CCCCAACACTCATCCATCCATCTTGAGCTACTTTTATGAAAC |
| | | | | | | | | CTTGGGGTTGAATTCTAAGCTCTTT |
| | | | | | | | | (SEQ ID NO: 24) |
| common_ | 67286863 | 2 | G | A | 0.127 | 0.155 | NA | CTGTCCCATAGCTTACTACTCCAGATCAGAAAAACCAGGTGT |
| 4979 | | | | | | | | GAGCTACAAATACTACCCAACTGTGAAAGAGTTGGCTGCCA |
| | | | | | | | | ACTCCGACATTTTGGTGGTTGCTTGTGCACTCACTGAGGAA |
| | | | | | | | | ACCCGCCACATTGTCAACCGTGAAGTCATCGACGCATTGGG |
| | | | | | | | | CACAAAGGGTGTTCTCATCAACATCGGGAGGGGTCC[C/T]CA |
| | | | | | | | | TGTCGACGAACCTGAGCTAGTATCAGCCCTGGTTGAAGGCC |
| | | | | | | | | GATTAGGGGGCGCTGGCCTTGATGTCTACCAAAATGAGCCT |
| | | | | | | | | GAGGTTCCTGAGGAGCTATTTGGTCTTGAAAACGTTGTCCTT |
| | | | | | | | | TTGCCTCATGTTGGAAGTGGCACTATCGAAACACGCCAGGC |
| | | | | | | | | CATGGCTGATCTGGTGGTTGGTAACCTTGAAGCT |
| | | | | | | | | (SEQ ID NO: 25) |
| common_ | 67370968 | 2 | A | G | NA | 0.155 | 0.127 | TGTGATTTGAAAACCAGAAGTGTTGTTGGATCTCAAC |
| 4980 | | | | | | | | ACAGAAATGATTTGTGGGTCTGTGTGTTCACCAAAC |
| | | | | | | | | CCAATCAAATTCCTACCACTCAAAGCTCCAAGCTCTG |
| | | | | | | | | GACATGGTGGATAATGATTGAGTCTGAAACACGAGT |
| | | | | | | | | CACTTTTCTCATCACTCAACATTTTACTCAGTACATTT |
| | | | | | | | | CTCGGTTCAATTCTTAA[T/C]CCATCAGCCATTAATTC |
| | | | | | | | | AAGTATCTCAAACGACATCGTTTTAACAGCCGTTATA |
| | | | | | | | | TATTTCTCCACCGCCGAACTATTCAAAAACGACAACC |
| | | | | | | | | AATTAGTCAAAACGACAACCAATATCAAAAACAGAGT |
| | | | | | | | | AAAAAAAAAAAATGAAAAAACAGAGTTTTTATTTTACC |
| | | | | | | | | GGAAAATTTCAGGATTTTCTCGGAAAGTGAAGAGG |
| | | | | | | | | (SEQ ID NO: 26) |

The inventors reasoned that, because leaf and flower percent total varin cannabinoid to total cannabinoid are correlated (FIG. 2), plants with alleles homozygous for predicting high-varin at both qtIV1 and qtIV2 might display a stronger high-varin phenotype than each allele alone. Within population GID:21 001 800, individuals were identified having both sets of homozygous alleles that predict the high-varin trait. When both predictive homozygous alleles were present, the average percent total varin cannabinoid/total cannabinoid ([CBDVA]+[THCVA]+[THCV]+[CBDV]/[CBDVA]+[THCVA]+[THCV]+[CBDV]+[CBCA]+[CBC]+[CBG]+[CBGA]+[CBDA]+[CBD]+[d9-THC]+[d8-THC]+[THCA]+[CBN])*100) was 2.6 fold higher in leaf tissue and 1.35 fold higher in flower than with the predictive homozygous alleles alone.

### EXAMPLE 3

### Developing KASP Markers for Detection of High Varin Trait in qtIV2

The high varin trait was introduced from a high varin donor plant GID: 20 000 110 0000 into a low varin acceptor plant 20 000 020 0000 and the progeny were selfed to generate an F2 population GID:21 002 059. These plants were carefully evaluated for the high varin trait by CA. The population is segregating for the high varin trait. KASP markers were used to validate the polymorphisms statistically associated with the high varin trait from the GWAS, narrow down the size of the QTL, and show the trait can be transferred into a low varin acceptor plant.

DNA was extracted for the KASP-assay using the QuickExtract Plant DNA Extraction Solution from LGC Genomics. The extraction was performed following the manufacturer's guideline with additional grinding as detailed in Example 2.

According to the QTL determination, Kompetitive allele-specific PCR (KASP) markers were designed on single nucleotide polymorphisms (SNPs) of the targeted loci and they were distributed over the genomic region. The loci are flanked by the SNPs from the QTL analysis, between them several additional SNPs were selected for KASP Markers and the KASP Markers incorporate the targeted SNP, which enables bi-allelic scoring of the SNP of interest. KASP primers for the assay were designed at LGC Genomics.

The KASP Assay mix contains three assay-specific non-labeled oligos: two allele-specific forward primers and one common reverse primer. The allele-specific primers each harbor a unique tail sequence that corresponds with a universal FRET (fluorescence resonant energy transfer) cassette; one labeled with FAM™ dye and the other with HEX·8 dye. The KASP Master mix contains the universal FRET cassettes, ROX™ passive reference dye, taq polymerase, free nucleotides, and MgCl₂ in an optimized buffer solution. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is no longer quenched and emits fluorescence. Bi-allelic discrimination is achieved through the competitive binding of the two allele-specific forward primers. If the genotype at a given SNP is homozygous, only one of the two possible fluorescent signals will be generated. If the genotype is heterozygous, a mixed fluorescent signal will be generated.

As the fluorescent signals are generated at the end of the thermal cycling and as those signals are clustered, three allelic groups can be differentiated: homozygous for Allele 1, heterozygous, and homozygous for Allele 2.
Genotypic and phenotypic data were used for a localized QTL mapping using R (v 4.0.0) with the R/qtl package (Broman et al., 2003). Initially, 82 KASP markers were trialed, of these only 27 were functional in our assay. Finally, 8 KASP markers were decided on for use in the construction of a genetic map. Markers were grouped in LGs with the formLinkageGroups function with a maximum recombination rate of 0.35 and minimum —log10 (p-value) logarithm of odds (LOD) threshold of 6. Marker order and genetic distances were established using the Kosambi mapping function [d=(1/4) In (1+2r/1−2r)], where d is the mapping distance and r is the recombination frequency (Kosambi, 1943). QTL mapping was carried out using the scan.cim function and a LOD of 3 was set as the QTL significance threshold. The KASP markers KASP_139, KASP_145 KASP_147, and KASP_151 were shown to be effective in distinguishing the high varin trait based either on the homozygous or heterozygous allele state (Tables 3 and Table 4). In particular KASP_145 and KASP_147 had the highest LOD scores in our assay and showed the ability to distinguish the high varin trait in all populations tested, including GID: 21 002 058. Fine mapping based on the KASP markers indicated that qtIV2 could be assigned to a region between KASP_139 and KASP_151 at position 68296752 - 70000000 on NC_044378.1 of the CS10 genome. The results of the marker panel assay in Table 1 and Table 2, together with this KASP marker data in Table 3 and Table 4, indicate the trait is a dominant Mendelian trait as the heterozygous state shows an intermediate increase in percent total varin compared to the homozygous allele states.

TABLE 3
KASP Markers shown to be effective in distinguishing the high
varin trait conferred by qtlV2 on chromosome NC_044378.1.
The presence of the high-varin trait is predicted by
the occurrence of the predictive allele for high varin
(predictive allele) using said markers, with the reference
allele being the allele at the same position in the
cs10 reference genome as described herein. The sequence
of the region of interest is also provided for context.

| | Position | | | | | | |
|---|---|---|---|---|---|---|---|
| | on | | | | | | |
| | cs10 | | Predictive | | | | Sequence of the |
| | of | | Allele for | | Primer Seq | | region of |
| | target | Ref | high | Primer_ | Varin | Primer Seq | interest |
| Name | SNP | Allele | varin | Common | Allele | Ref | (100 bp) |
| PG_ | 65305636 | G | T | GCGGAGTTTGGATTT | TAACTCAACCCTACG | CTCAACCCTACGATT | GGCGGATGTGGGCGG |
| KASP_ | | | | GAAGGTGGAA | ATTCGCCAAA | CGCCAAC | AGTTTGGATTTGAAG |
| 127 | | | | (SEQ ID NO: 27) | (SEQ ID NO: 28) | (SEQ ID NO: 29) | GTGGAAAAAGTTGGA |
| | | | | | | | GAGGGA[G/T]TTGG |
| | | | | | | | CGAATCGTAGG |
| | | | | | | | GTTGAGTTATTATTG |
| | | | | | | | GTGGAGAATGGACGG |
| | | | | | | | TGGAGA |
| | | | | | | | (SEQ ID NO: 30) |
| PG_ | 66465699 | G | A | GCAGCTCATTGAGAT | TGTGGATGCTTCCAT | TGGATGCTTCCATGG | ATATGTGTAGGCTAT |
| KASP_ | | | | GACACCCAA | GGTCGTACA | TCGTACG | CATTGTGTCATGCTG |
| 133 | | | | (SEQ ID NO: 31) | (SEQ ID NO: 32) | (SEQ ID NO: 33) | TGGATGCTTCCATGG |
| | | | | | | | TCGTAC[G/A]TTGG |
| | | | | | | | GTGTCATCTCA |
| | | | | | | | ATGAGCTGCGACAAT |
| | | | | | | | GAGGCAA [symbol] |
| | | | | | | | (SEQ ID NO: 34) |
| PG_ | 67298055 | G | A | AGTGACATTGGATTG | TAGAAAGAGGGTAC | TAGAAAGAGGGTAC | TGGTGGTTCCATTAT |
| KASP_ | | | | ATCATTCTGCGAATC | CACTGCCAT | CACTGCCAC | TAGTGACATTGGATT |
| 136 | | | | ACTGCCAT | (SEQ ID NO: 36) | (SEQ ID NO: 37) | GATCATTCTGCGAAT |
| | | | | (SEQ ID NO: 35) | | | CAGTGG[G/A]TGGC |
| | | | | | | | AGTGGTACCCT |
| | | | | | | | CTTTCTACCAAACTT |
| | | | | | | | GGCATCATAAAACAT |
| | | | | | | | TTTAAA |
| | | | | | | | (SEQ ID NO: 38) |
| PG_ | 68204540 | A | G | CCTTCGGAATCAAGG | CTGTTTCTTTTGGCA | CCTGTTTCTTTTGGC | TTGTTTTGAGATTTT |
| KASP_ | | | | AGAAGGATGTT | GGAGGCG | AGGAGGCA | TAATTTTTCGTTTGC |
| 138 | | | | (SEQ ID NO: 39) | (SEQ ID NO: 40) | (SEQ ID NO: 41) | CTGTTTCTTTTGGCA |
| | | | | | | | GGAGGC[A/G]TGCC |
| | | | | | | | CGTCTGTGAAA |
| | | | | | | | AACATCCTTCTCCTT |
| | | | | | | | GATTCCGAAGGAAAG |
| | | | | | | | CGTGTT |
| | | | | | | | (SEQ ID NO: 42) |
| PG_ | 68296752 | T | C | GACATATGGGATGTG | TCATTTTTGTTGTTT | GCTATCATTTTTGTT | AGATATTTGGTTGTG |
| KASP_ | | | | GATGTTTGGGAA | CGAAATGAAACTTTC | CTTTCAA | TTGGATGACATATGG |
| 139 | | | | (SEQ ID NO: 43) | GTTTCGAAATGAAAA | (SEQ ID NO: 45) | GATGTGGATGTTTGG |
| | | | | | G | | GAAGCA[T/C]TGAAA |
| | | | | | (SEQ ID NO: 44) | | GTTTCATTTCGAAACA |
| | | | | | | | ACAAAAATGATAGCC |
| | | | | | | | GAGTAATGATCACAA |
| | | | | | | | (SEQ ID NO: 46) |
| PG_ | 68871752 | C | T | CACAAGAGGTACAAC | TTCTTAAACTGTTTA | CTTAAACTGTTTAGT | AACGGTAGGAGAAAA |
| KASP_ | | | | AACCACAACCAT | GTGATCAATTGATGG | GATCAATTGATGGG | CCCGGAACCACAAGA |
| 145 | | | | (SEQ ID NO: 47) | A | (SEQ ID NO: 49) | GGTACAACAACCACA |
| | | | | | (SEQ ID NO: 48) | | ACCATC[C/T]CCAT |
| | | | | | | | CAATTGATCAC |
| | | | | | | | TAAACAGTTTAAGAA |
| | | | | | | | CTAATGAGATATCTG |
| | | | | | | | ATGATG |
| | | | | | | | (SEQ ID NO: 50) |
| PG_ | 69455923 | C | T | GGAGTACTCTTATCT | GTTTTCATAGTTTTA | ATAACACTTACATCT | AAAAAAAAGCTATCA |
| KASP_ | | | | ATAACACTTACATCT | TTT | GTTTTCATAGTTTTA | TAATGTTTTCATAGT |
| 147 | | | | TTTGGATCAAGCAT | (SEQ ID NO: 52) | TTC | TTTATGCTTGATCCA |
| | | | | (SEQ ID NO: 51) | | (SEQ ID NO: 53) | AAAGATAAGAGTACT |
| | | | | | | | CCATAATAACACTTA |
| | | | | | | | CATCTTT[C/T]CCA |
| | | | | | | | TGTTGG |
| | | | | | | | ATTCTTCACAA |
| | | | | | | | (SEQ ID NO: 54) |
| PG_ | 70024415 | C | T | TCACCTGAGGGATT | CAGTGAAGCAAACTA | AGTGAAGCAAACTAA | AATGACGCGAATTGA |
| KASP_ | | | | TCCGCAACATA | ATCCTCGTCAA | TCCTCGTCAG | GGTTTCCATACTCAC |
| 151 | | | | (SEQ ID NO: 55) | (SEQ ID NO: 56) | (SEQ ID NO: 57) | CTGAGGGATTTCCGC |
| | | | | | | | AACATA[C/T]TGA |
| | | | | | | | CGAGGATTAGTT |
| | | | | | | | TGCTTCACTGACAAT |
| | | | | | | | TGACAATCCTAATTC |
| | | | | | | | AACACA |
| | | | | | | | (SEQ ID NO: 58) |

[symbol]

indicates data missing or illegible when filed

TABLE 4
KASP Marker data - KASP markers KASP_139, KASP_145 KASP_147,
and KASP_151 were shown to be effective in distinguishing the high varin
trait conferred by qtlV2 on chromosome NC_044378.1, based either
on the homozygous or heterozygous allele state (denoted by an asterisk
(*)). The mean percent total varin (%) is provided for plants homozygous
for Allele 1 (Allele 1), homozygous for Allele 2 (Allele 2) or heterozygous
for the alleles (Hetero) detected by the markers.

| | | Position | | Variance | | | |
|---|---|---|---|---|---|---|---|
| Marker | Position | on cs10 | | explained | Allele | Allele | |
| name | (cM) | (bp) | LOD | (%) | 1 | 2 | Hetero |
| KASP_127 | 0 | 65305636 | 0.29 | 8.5 | 21.74 | 33.57 | 27.57 |
| KASP_133 | 3.889359 | 66465699 | 0.39 | 12 | 20 | 37.12 | 27.77 |
| KASP_136 | 9.300948 | 67298055 | 0.02 | 18.76 | 18.61 | 36.96 | 29.72 |
| KASP_138 | 17.275199 | 68204540 | 0.31 | 27.5 | 15.88 | 38.06 | 29.89 |
| KASP_139* | 18.733199 | 68296752 | 4.09 | 30.3 | 15.62 | 38.85 | 30.46 |
| KASP_145* | 22.499733 | 68871752 | 8.51 | 45.7 | 12.16 | 39.93 | 32.89 |
| KASP_147* | 22.499743 | 69400000 | 8.51 | 45.7 | 12.16 | 39.93 | 32.89 |
| KASP_151* | 24.710048 | 70024415 | 3.67 | 44.47 | 12.22 | 42.13 | 30.22 |

Sequencing primers were designed for each of the SNPs in Table 1 and Table 2. Briefly, primers were designed to amplify the region containing the SNP for subsequent sequencing of the region to determine whether one or more allele associated with the high varin trait is present, defining qtIV1 and/or qtIV2, in the plant (Table 5 and Table 6).

TABLE 5
Sequencing primers for detection of alleles associated with high varin content in
qtIV1.

| QTLV1 SNP | Primer 1 Fw | Primer 1 Rv | Primer 2 Fw | Primer 2 Rv |
|---|---|---|---|---|
| GBScompat_ | ATGTGGTGTGTT | TTTCAAGTGGAC | ATGTGGTGTGTTC | TCAAGTGGACAAT |
| common_353 | CCCAAGCA | AATGGGGT | CCAAGCA | GGGGTAC |
| | (SEQ ID NO: 59) | (SEQ ID NO: 60) | (SEQ ID NO: 61) | (SEQ ID NO: 62) |
| common_1811 | CATCTTCGCCTC | TGAGCTCCCATT | ATCTTCGCCTCCAC | TGAGCTCCCATTC |
| | CACCAGTC | CGAGGATG | CAGTCT | GAGGATG |
| | (SEQ ID NO: 63) | (SEQ ID NO: 64) | (SEQ ID NO: 65) | (SEQ ID NO: 66) |
| common_2008 | ACCTGGGCAAG | AGAGGGAACAA | CCTGGGCAAGTTT | AGAGGGAACAAC |
| | TTTCAGATTTG | CCACACACA | CAGATTTGT | CACACACA |
| | (SEQ ID NO: 67) | (SEQ ID NO: 68) | (SEQ \| DNO: 69) | (SEQ ID NO: 70) |
| GBScompat_ | TTTGCAGCAGA | TCCCGTGATGA | TCGGAGTTGGACC | TCCCGTGATGAAA |
| common_374 | GTAGCCCTC | AATGCAGCT | AAGATGA | TGCAGCT |
| | (SEQ ID NO: 71) | (SEQ ID NO: 72) | (SEQ ID NO: 73) | (SEQ ID NO: 74) |
| common_1813 | TTCCGGTGTAT | TCCATACCAGA | TCCGGTGTATGAA | TCCATACCAGAGG |
| | GAAGGTGGA | GGTTCTGCTC | GGTGGAA | TTCTGCTC |
| | (SEQ ID NO: 75) | (SEQ ID NO: 76) | (SEQ ID NO: 77) | (SEQ ID NO: 78) |
| common_1939 | TCAGATTAAAGC | GCAGCTTTGAA | GATTAAAGCCGGT | GCAGCTTTGAAAA |
| | CGGTGCCA | AACCCTCTCG | GCCAAGC | CCCTCTCG |
| | (SEQ ID NO: 79) | (SEQ ID NO: 80) | (SEQ ID NO: 81) | (SEQ ID NO: 82) |
| common_2007 | TAAGCCCAATG | TTCAGGCATTCC | GCCATTAAGCCCA | TTCAGGCATTCCA |
| | CCTCCATGG | AAGTGGGA | ATGCCTC | AGTGGGA |
| | (SEQ ID NO: 83) | (SEQ ID NO: 84) | (SEQ ID NO: 85) | (SEQ ID NO: 86) |
| common_2060 | TGGCTTGTTTTG | CCAAGTACTGT | TGGCTTGTTTTGTG | AAGTACTGTGCCA |
| | TGTTTGGTGT | GCCACCCAA | TTTGGTGT | CCCAAGG |
| | (SEQ ID NO: 87) | (SEQ ID NO: 88) | (SEQ ID NO: 89) | (SEQ ID NO: 90) |
| common_1758 | TGCATTCTCCGT | GGGATACAACT | AGAGTTCGCTGCA | CCAGCGAATGGT |
| | GGTTTGGA | CTTTCCAGCGA | TTCTCCG | CAAGATGTC |
| | (SEQ ID NO: 91) | (SEQ ID NO: 92) | (SEQ ID NO: 93) | (SEQ ID NO: 94) |
| common_1940 | AGATTATCCACC | CCAGCACCACC | GATTATCCACCGC | CCAGCACCACCC |
| | GCCACAGC | CACAATGTA | CACAGCT | ACAATGTA |
| | (SEQ ID NO: 95) | (SEQ ID NO: 96) | (SEQ ID NO: 97) | (SEQ ID NO: 98) |
| common_1777 | CCAAGCAGGCT | CCACAATCCCAT | AGCAGGCTTTTCAA | CCACAATCCCATC |
| | TTTCAAGGG | CCACACCA | GGGTGA | CACACCA |
| | (SEQ ID NO: 99) | (SEQ ID NO: 100) | (SEQ ID NO: 101) | (SEQ ID NO: 102) |
| GBScompat_ | GACCGGAGTTT | TTCAGCGAGCT | ACCGGAGTTTCAG | TTCAGCGAGCTTC |
| common_346 | CAGCTCGAT | TCTCCTTCA | CTCGATC | TCCTTCA |
| | (SEQ ID NO: 103) | (SEQ ID NO: 104) | (SEQ ID NO: 105) | (SEQ ID NO: 106) |
| common_1735 | ATCATCCCCAC | CATCACCATCAC | AGATCATCCCCAC | CATCACCATCACC |
| | CTCGAGAGT | CACCAGCT | CTCGAGA | ACCAGCT |
| | (SEQ ID NO: 107) | (SEQ ID NO: 108) | (SEQ ID NO: 109) | (SEQ ID NO: 110) |
| common_1746 | CACCAGGCAAA | ATCTTGTTGGCA | CTGGAACACCAGG | ATCTTGTTGGCAG |
| | CACCAATGG | GAGGAGGG | CAAACAC | AGGAGGG |
| | (SEQ ID NO: 111) | (SEQ ID NO: 112) | (SEQ ID NO: 113) | (SEQ ID NO: 114) |
| common_1945 | TGCCAGAAATG | TTGGAGGATTC | GCCAGAAATGTTG | TTGGAGGATTCCC |
| | TTGCCCATG | CCCATTGGC | CCCATGT | CATTGGC |
| | (SEQ ID NO: 115) | (SEQ ID NO: 116) | (SEQ ID NO: 117) | (SEQ ID NO: 118) |
| common_2000 | ACCGTGCTCCA | CCATTGGAGCC | ACCGTGCTCCATG | TGGAGCCAGCAA |
| | TGTATAGCT | AGCAACAAG | TATAGCT | CAAGAACT |
| | (SEQ ID NO: 119) | (SEQ ID NO: 120) | (SEQ ID NO: 121) | (SEQ ID NO: 122) |
| common_1987 | TTTCACAACAAA | TGAATCGCACA | TCACAACAAAAAGA | TGAATCGCACAAG |
| | AAGACACGAGA | AGAGCCCAT | CACGAGAAA | AGCCCAT |
| | (SEQ ID NO: 123) | (SEQ ID NO: 124) | (SEQ ID NO: 125) | (SEQ ID NO: 126) |
| rare_214* | GCTTCCTGCAA | TCCGACTGTGG | AGCACCAAGTATAA | AAGCCTCTTCTGT |
| | ATTATAGCACCA | AGCCTAGTT | AGAACGCA | TGCCTCC |
| | (SEQ ID NO: 127) | (SEQ ID NO: 128) | (SEQ ID NO: 129) | (SEQ ID NO: 130) |
| common_1780* | CATCCGACACT | GGCGCCAACCC | CCATCCGACACTC | GGCGCCAACCCA |
| | CCTCAACGT | AATTGTATC | CTCAACG | ATTGTATC |
| | (SEQ ID NO: 131) | (SEQ ID NO: 132) | (SEQ ID NO: 133) | (SEQ ID NO: 134) |

TABLE 6
Sequencing primers for detection of alleles associated
with high varin content in qtIV2.

| QTLV2 SNP | Primer 1 Fw | Primer 1 Rv | Primer 2 Fw | Primer 2 Rv |
|---|---|---|---|---|
| common_ | TGAAGAGCCA | TGAGAGGAAC | GAGCCATGGT | TGAGAGGAAC |
| 5002 | TGGTGGGATC | CAAAGCATGA | GGGATCTTGG | CAAAGCATGA |
| | (SEQ ID | GT | (SEQ ID | GT |
| | NO: 135) | (SEQ ID | NO: 137) | (SEQ ID |
| | | NO: 136) | | NO: 138) |
| pooled | TGCTTCCAAG | GAACTGGTAC | TGCTTCCAAG | AACTGGTACC |
| Seq_7 | AAACCAGTCA | CGCCTCATGT | AAACCAGTCA | GCCTCATGTC |
| | (SEQ ID | (SEQ ID | (SEQ ID | (SEQ ID |
| | NO: 139) | NO: 140) | NO: 141) | NO: 142) |
| common_ | AACACTGGGG | TGAGAGTGGA | CACTGGGGAT | TGAGAGTGGA |
| 4995 | ATTTACCGGG | AAGCACTGGA | TTACCGGGTT | AAGCACTGGA |
| | (SEQ ID | C | (SEQ ID | C |
| | NO: 143) | (SEQ ID | NO: 145) | (SEQ ID |
| | | NO: 144) | | NO: 146) |
| common_ | TCAGTGCCAA | AAATCGCAAG | AGTGCCAACT | AAGCAGAGTT |
| 4973 | CTTGGTCACC | CAGAGTTGGC | TGGTCACCAA | GGCACTGAGG |
| | (SEQ ID | (SEQ ID | (SEQ ID | (SEQ ID |
| | NO: 147) | NO: 148) | NO: 149) | NO: 150) |
| common_ | AGGCATCAGG | TGGATGAGTG | GGCATCAGGA | GGATGGATGA |
| 4981 | AAGGTGATCA | TTGGGGGAGA | AGGTGATCAT | GTGTTGGGGG |
| | (SEQ ID | (SEQ ID | CT | (SEQ ID |
| | NO: 151) | NO: 152) | (SEQ ID | NO: 154) |
| | | | NO: 153) | |
| common_ | GCTTGTGCAC | CCAACCACCA | GCTTGTGCAC | AACCACCAGA |
| 4979 | TCACTGAGGA | GATCAGCCAT | TCACTGAGGA | TCAGCCATGG |
| | (SEQ ID | (SEQ ID | (SEQ ID | (SEQ ID |
| | NO: 155) | NO: 156) | NO: 157) | NO: 158) |
| common_ | TGTGGGTCTG | TGAATAGTTC | GTGGGTCTGT | TGAATAGTTC |
| 4980 | TGTGTTCACC | GGCGGTGGAG | GTGTTCACCA | GGCGGTGGAG |
| | (SEQ ID | (SEQ ID | (SEQ ID | (SEQ ID |
| | NO: 159) | NO: 160) | NO: 161) | NO: 162) |

### EXAMPLE 4

### Identification of Candidate Genes

An in silico analysis allowed for the annotation of the identified QTLs with putative candidate genes encoded in the region.

The region on Chromosome NC_044373.1 starting at position 10-20,000,000 centered around the SNP GBScompat_common_353 with the highest LOD score was searched for all candidate genes in this region based on the CS10 genome annotation.

This region comprised 267 genes. From these a candidate gene was identified, LOC115712547, from the annotated CS10 gene list, based on its likely involvement in the biosynthesis of hexanoyl-CoA and its proximity to GBScompat_common_353. LOC115712547 is annotated to be a protein that is a member of acyl-activating enzyme superfamily, named 4-coumarate--CoA ligase-like 1. Members of this family can potentially form hexanoyl-CoA, disruption of function or normal behavior of this protein could lead to the high-varin phenotype.

The QTL on Chromosome NC_044378.1 was evaluated using the same approach for all genes found between 65,000,000 and 71,228,646. This region comprised 457 genes. In this case, to identify the involved biochemical pathways of the candidate genes, the inventors used Pannzer2 (Petri Toronen, Alan Medlar, and Liisa Holm (2018) PANNZER2: a rapid functional annotation web server) in combination with the KEGG (Kanehisa & Goto (2000) KEGG: Kyoto Encyclopedia of Genes and Genomes). Eighteen genes on this QTL are predicted to be involved in biochemical pathways by the described approach. Amongst these, a cluster of seven candidate genes was identified, LOC115697567, LOC115697560, LOC115697568, LOC115697574, LOC115697562, LOC115697566, LOC115696799, due to their proximity to common_5002 the SNP at qtIV2 with the highest LOD score and because of their predicted enzymatic function (Table 7). All seven candidate genes are predicted to encode GDSL-type lipases, these proteins have roles in the degradation of fatty acids. Fatty acid degradation can impact the percent total varin to non-varin cannabinoids by altering the of available butonyl-CoA to hexonyl Co-A. Loss of or alteration of one or all these candidates, or in various combinations, could cause the high-varin trait at qtIV2.

In a comparative analysis, the reactions catalysed by each of the enzymes predicted to be involved in the production of varin cannabinoids were characterized by their reaction codes using databases such as the Kyoto Encyclopedia of Genes and Genomes (KEGG; genome.jp). These reaction codes were compared to the reactions predicted for the genes identified within the qtIV1 and qtIV2. In this analysis there was no correlation between the reactions, suggesting at least for qtIV2, a novel mode of action with respect to the production of varin cannabinoids. This comparative approach did not identify the 4-coumarate--CoA ligase-like 1 identified for qtIV1.

A manual inspection of the genes in qtIV2 identified several additional candidate genes that are predict based on their NCBI annotation. The acyl-acyl carrier proteins are predicted to be involved in pathways that may influence the relative amount of precursor hexonyl-CoA or butonyl-CoA. Oxysterol binding protein may be involved in binding sterol or lipid like small molecules for transport impacting substrate availability of putative precursors like hexonyl-CoA or butonyl-CoA (Table 7).

TABLE 7
Candidate genes identified within the QTL on chromosome NC_044378.1
(qtlV2) based on their proximity to common_5002 the SNP at qtlV2 with
the highest LOD score and because of their predicted enzymatic function.

| | | | KEGG | | Start | End |
|---|---|---|---|---|---|---|
| Gene | LOC | XP | ID | Pathway | Position | Position |
| Lipase_GDSL | 115697567 | XP_030480495.1 | R00630 | Carboxylic-ester | 68940361 | 68944336 |
| | | | | hydrolase | | |
| Lipase_GDSL | 115697560 | XP_030480488.1 | R00630 | Carboxylic-ester | 68955855 | 68958528 |
| | | | | hydrolase | | |
| Lipase_GDSL | 115697568 | XP_030480496.1 | R00630 | Carboxylic-ester | 68968188 | 68970354 |
| | | | | hydrolase | | |
| Lipase_GDSL | 115697574 | XP_030480503.1 | R00630 | Carboxylic-ester | 68974485 | 68977069 |
| | | | | hydrolase | | |
| Lipase_GDSL | 115697562 | XP_030480490.1 | R00630 | Carboxylic-ester | 68983864 | 68987448 |
| | | | | hydrolase | | |
| Lipase_GDSL | 115697566 | XP_030480494.1 | R00630 | Carboxylic-ester | 68996304 | 69000685 |
| | | | | hydrolase | | |
| Lipase_GDSL | 115696799 | XP_030479543.1 | R00630 | Carboxylic-ester | 69013928 | 69027277 |
| | | | | hydrolase | | |
| acyl-acyl | 115697587 | XP_030480523.1 | NA | Fatty acid | 69247325 | 69249937 |
| carrier | | | | synthesis | | |
| protein | | | | | | |
| acyl-acyl | 115697585 | XP_030480521.1 | NA | Fatty acid | 69253376 | 69257044 |
| carrier | | | | synthesis | | |
| protein | | | | | | |
| acyl-acyl | 115697580 | XP_030480512.1 | NA | Fatty acid | 69286245 | 69290147 |
| carrier | | | | synthesis | | |
| protein | | | | | | |
| Oxysterol- | 115696214 | XP_030478978.1 | NA | Sterol transport | 69451790 | 69461849 |
| binding | | | | | | |
| protein | | | | | | |

1.-34. (canceled)

35. A method for identifying a Cannabis sativa plant comprising in its genome a high-varin QTL, the method comprising the steps of:
(i) providing a population of Cannabis plants;
(ii) genotyping at least one plant from the population by detecting an allele of one or more polymorphisms associated with a high-varin trait as defined in Table 1 or Table 2; and
(iii) identifying one or more plants containing the high-varin QTL.

36. The method of claim 35, further comprising the steps of:
(iv) crossing the plant containing the high-varin QTL of step (iii) with at least one recipient parent plant that does not have the high-varin QTL to obtain a progeny population of Cannabis plants;
(v) genotyping at least one plant from the progeny population with respect to the high-varin QTL by detecting the allele of the one or more polymorphisms associated with the high-varin trait as defined in Table 1 or Table 2; and
(vi) selecting one or more progeny plants having the high-varin QTL.

37. The method of claim 36, further comprising the steps of:
(vii) crossing the one or more progeny plants with the plant containing the high-varin QTL of step (iii); or
(viii) selfing the one or more progeny plants.

38. The method of claim 35, wherein the polymorphisms in Table 1 define a first high-varin QTL associated with the high-varin trait in the Cannabis sativa plant and the polymorphisms in Table 2 define a second high-varin QTL associated with the high-varin trait in the Cannabis sativa plant.

39. The method of claim 38, wherein the identified plant and/or the progeny plant contains the first high-varin QTL and the second high-varin QTL.

40. The method of claim 39, wherein the identified plant and/or the progeny plant displays the high-varin trait.

41. The method of claim 36, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

42. The method of claim 41, wherein the molecular markers are KASP molecular markers comprising one or more primer pairs as defined in Table 3.

43. The method of claim 41, wherein the molecular markers are for detecting polymorphisms at regular intervals within the QTL such that recombination can be excluded.

44. The method of claim 41, wherein the molecular markers are for detecting polymorphisms at regular intervals within the QTL such that recombination can be quantified to estimate linkage disequilibrium between a particular polymorphism and a high-varin phenotype.

45. The method of claim 36, wherein the recipient parent plant has one or more desirable characteristics unrelated to varin content and wherein the one or more progeny plants having the high-vain QTL has the one or more desirable characteristics unrelated to vain content.

46. The method of claim 35, wherein the high-vain QTL is selected from:
i. a quantitative trait locus having a sequence that corresponds to nucleotides 5139731 to 47648106 of NC_044373.1 of the CS10 genome and contains an allele of one or more polymorphisms associated with the high-varin trait as defined in Table 1, or a genetic marker linked to the QTL; and/or
ii. a quantitative trait locus having a sequence that corresponds to nucleotides 68296752 to 70024415 of NC_044378.1 of the CS10 genome and contains an allele of one or more polymorphisms associated with the high vain trait as defined in Table 2, or a genetic marker linked to the QTL.

47. A Cannabis sativa plant obtained according to the method of claim 36.

48. The Cannabis sativa plant of claim 47, wherein the plant contains a first high-varin QTL characterized by an allele of one or more polymorphisms associated with a high-vain trait as defined in Table 1 and a second high-varin QTL characterized by an allele of one or more polymorphisms associated with a high-varin trait as defined in Table 2.

49. A plant extract obtainable from a Cannabis sativa plant of claim 47.

50. An isolated gene that controls a high-varin trait in a Cannabis sativa plant, wherein the gene corresponds to LOC115712547 with reference to the CS10 genome, and which encodes a 4-coumarate--CoA ligase-like 1 protein.
